(19) United States
(12) Patent Application Publication
(10) Pub. No.: US 2023/0193289 A1
(43) Pub. Date: Jun. 22, 2023

(54) COMPOSITIONS AND METHODS FOR TREATING FABRY DISEASE

(71) Applicant: Taipei Veterans General Hospital, Taipei City (TW)

(72) Inventors: Dau-Ming Niu, Taipei City (TW); Yu-Ying Lu, Changhua County (TW); Yen-Fu Cheng, Taipei City (TW); Yun-Ru Chen, Taipei City (TW); Ching-Tzu Yen, Taipei City (TW); Chun-Ying Huang, New Taipei City (TW)

(73) Assignee: Taipei Veterans General Hospital, Taipei City (TW)

(21) Appl. No.: 17/817,657

(22) Filed: Aug. 5, 2022

Related U.S. Application Data
(60) Provisional application No. 63/230,072, filed on Aug. 6, 2021.

(51) Int. Cl. C12N 15/52; C12N 9/22; C12N 15/63; A61P 25/00

(52) U.S. Cl. CPC C12N 15/52; C12N 9/22; C12N 15/63; A61P 25/00; C12N 2310/20

(57) ABSTRACT

A composition for treating Fabry disease, which includes a polynucleotide having gene editing function for correction of the specific mutation (IVS4+919 G>A) of GLA gene. A method for treating Fabry disease, which uses gene editing systems for correction of the specific mutation (IVS4+919 G>A) of GLA gene in Fabry disease.

This application claims the benefit of U.S. Provisional Patent Application No. 63/230,072 filed on Aug. 6, 2021, which is incorporated by reference herein in its entirety.

This application contains a Sequence Listing XML, the file name is 3942_SEQ List-0804, created on Aug. 4, 2022, the size is 57 KB, which is incorporated herein by reference.

### FIELD OF THE INVENTION

The present invention relates to gene correction of point mutation in intron 4 (GLA IVS4+919G>A) of the GLA gene in Fabry disease as a gene therapy strategy for Fabry disease by using a gene editing technology based on Clustered Regularly Interspaced Short Palindromic Repeats (CRISPR)/Cas9 system or Adenine base editor (ABE).

### DESCRIPTION OF PRIOR ART

Fabry disease is an X-linked lysosomal storage disease, a rare hereditary disease, and the current global prevalence rate is approximately from 1: 40,000 to 1: 60,000. The disease is caused by mutations in the GLA gene located on the X chromosome, leading to abnormal metabolism and a large accumulation of globotriaosylceramide (Gb3) in the lysosomes of cells in various tissues due to a deficiency of alpha-galactosidase (GLA). The cells in various tissues include endothelial cells and smooth muscle cells in blood vessels, cardiomyocytes and valve fibroblasts in the heart, glomerulus and tubular epithelial cells in kidneys, reticulum cells in corneas and connective tissues, and ganglion cells of the nervous system, etc. The abnormal accumulation of Gb3 causes cell damage, leading to damage to organ functions, and causing pathological changes of the heart, kidneys, cerebral vessels and peripheral nerves.

GLA IVS4+919G>A (NM_00016: c.639+919G>A) is reported as a point mutation related to the late-onset cardiac variant Fabry disease. The point mutation is conversion of guanine (G) to adenine (A) at nucleotide 919 in intron 4 (IVS4) of the GLA gene. The point mutation causes aberrant mRNA alternative splicing, which causes a 57-nucleotide intronic sequence in intron 4 is mistakenly recognized as an exon and retained, resulting in a premature TGA stop codon between the fourth and the fifth exons and consequently translating truncated GLA proteins. When the aberrant alternative splicing patterns become the main splicing products in the cell, only few normal GLA mRNA splicing patterns remain, as a result, the enzyme activity of the GLA in the cell with IVS4+919G>A point mutation is about 5-10% of the normal level. Recent research reports show that the prevalence rate of this gene mutation among Taiwanese is extremely high, one in every 1,471 male newborns carry this gene point mutation, and the prevalence rate of females is as high as 1/750.

Currently, the main treatment method of Fabry disease is enzyme replacement therapy. Although it can effectively delay the disease status for most patients, it requires intravenous injection of protein enzyme drugs once every two weeks lifelong to maintain therapeutic effects. The high injection frequency not only causes inconveniences and deteriorates the quality of life for the patients, but also incurs expenses on health care resources because of the high price of protein enzyme drugs.

The gene editing technology can correct mutations in genes directly, allowing the cell itself to permanently produce functional GLA enzyme protein. The advent of CRISPR/Cas9 gene editing technology is a major breakthrough in the field of gene therapy. Cas9 is used to identify and cut the target double-stranded DNA (dsDNA), wherein Cas9 needs to be guided by a guide RNA (gRNA) to reach the target sequence for working, then cause double-strand breaks, and finally the DNA repair mechanism in the cell is used to achieve the gene reprogramming effect.

However, gene editing technology is evolving, different from traditional CRISPR/Cas9 technology that requires inducement of DNA double-strand breaks, base editing systems can correct point mutations more accurately. The base editing systems are mainly composed of SpCas9 nickase (for example, nCas9), gRNA and deaminase, in which a small nick is formed when the non-editing strand that is bound to nCas9 and gRNA undergoes single-strand cleavage, and a base editing window is formed in the target sequence, then base converting is occurred in the base editing window through deaminase. Compared to the traditional CRISPR/Cas9, the base editing system reduces the generation of insertion/deletion (indel) because the generation of DNA double-strand breaks is not required. In addition, it can effectively and accurately correct a single base without relying on exogenous DNA as a template. Furthermore, the use of nCas9 can also reduce the occurrence of off-target effects to increase the safety of disease treatments and clinical applications.

In the base editing system, an adenine base editor (ABE) can transvert A-T into G-C. When E. coli's TadA is used as deaminase, A can be effectively deaminated and transverted into Inosine (I), and then I is finally recognized as G by DNA polymerase in the DNA repair mechanism or replication process to achieve the effect of transverting A to G. However, when using the adenine base editor, any As in the base editing window may undergo base conversion, the so called a bystander effect, and it may cause unexpected mutations and risks. Thus, not all hereditary diseases can be treated by using an adenine base editor.

### SUMMARY OF THE INVENTION

The present invention provides a composition for treating Fabry disease, wherein the composition comprises a polynucleotide having a gene editing function for correcting a specific mutation (IVS4+919 G>A) of GLA gene in Fabry disease. The present invention further provides a method for treating Fabry disease by using a gene editing system to correct the specific mutation (IVS4+919 G>A) of GLA gene in Fabry disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are conceptual diagrams of the experimental design, which show the application of gene editing to patient cells carrying GLA IVS4+919G>A. FIG. 1A shows the application of the CRISPR/Cas9 system. When in a state of GLA IVS4+919G>A mutation, 57 nucleotides in intron 4 are inserted to cause an aberrant mRNA splicing pattern. Therefore, suitable sgRNAs are respectively designed upstream and downstream of the point mutation, double-strand breaks are generated in the GLA gene sequence through HiFi Cas9 and deletions are formed in a non-homologous end joining (NHEJ) manner. FIG. 1B shows the application of the adenine base editor (ABE). A sgRNA having a length of 20 nucleotides is designed for making the target site GLA IVS4+919G>A to fall into the sgRNA. The base editing conditions of GLA IVS4+919G>A, +918 and +920 are observed.

FIGS. 2A and 2B show experimental designs of applying the CRISPR/Cas9 system on fibroblasts of cardiac type Fabry disease carrying IVS4+919G>A and efficiency screening of sgRNA. FIG. 2A shows that the double-strand breaks are generated upstream and downstream of the 57-nucleotide through HiFi Cas9, that causes GLA mRNA aberrant splicing, and deletions are formed in a manner of non-homologous end joining, thereby altering the mRNA splicing patterns. FIG. 2B shows the principle of a surrogate reporter system. FIG. 2C shows the HiFi Cas9-sgRNA2+3 plasmid. FIG. 2D shows the sites in the GLA gene targeted by sgRNA2 and sgRNA3 and a 97 bp deletion is expected to be generated.

FIGS. 3A to 3F show the application of the CRISPR/Cas9 system to fibroblasts of the cardiac type Fabry disease carrying IVS4+919G>A. 48 hours after each plasmid simultaneously carrying HiFi Cas9 and sgRNA is transfected by electroporation into the fibroblasts of the cardiac type Fabry disease carrying IVS4+919G>A, treated with puromycin for 66 hours to screen out the successfully transfected cells. FIG. 3A shows the gene editing efficiencies observed with polymerase chain reactions; the fragment size that no deletions generated is 293 bp; the expected fragment size generated by deletions is 196 bp. FIG. 3B shows the analysis of gene editing efficiency by quantifying the gel images with Image J. FIG. 3C shows the quantitative analyses of normal and aberrant GLA mRNA splicing patterns carried out by quantitative real time polymerase chain reactions, in which beta-actin is used as the reference for normalization, and the expression level of the wild-type fibroblast is set as 1, and the untreated IVS4 cells are used as the control group. One-way ANOVA is used for analysis, and the differences between two groups are analyzed by the Student's t-test. The bar graph is presented as mean value ± standard deviation (mean±SD), * means p<0.05. FIG. 3D shows GLA protein expression levels analyzed by western blot, GAPDH is used as the reference for normalization. FIG. 3E shows the quantitative results of the GLA protein expression levels, and the expression level of the wild-type fibroblasts is set as 1. FIG. 3F shows the detected cellular GLA enzyme activity, and the enzyme activity of the wild-type fibroblasts is set as 100%, and the untreated IVS4 cells are used as the control group. One-way ANOVA is used for analysis, and the difference between two groups is analyzed by the Student's t-test. The bar graph is presented as mean value ± standard deviation (mean±SD), * means p<0.05. WT: wild type; ns: no significant difference.

FIGS. 4A to 4E show the applications of the adenine base editing system to IVS4+919G>A in the fibroblasts of the cardiac type Fabry disease.

FIG. 4A shows that two sgRNAs are designed in the GLA genome for making the target site IVS4+919G>A to fall into the base editing window, wherein sgRNA5 can make the target to fall into the 4th position of the protospacer sequence, and sgRNA6 makes the target to fall into the 5th position. The underlined region is the splice-donor site. FIG. 4B shows the ABEmax-sgRNA5 and ABEmax-sgRNA6 plasmids. FIG. 4C shows that SpCas9 is stained by immunofluorescence and the cell nuclei are localized with DAPI. FIG. 4D shows that the plasmid of ABEmax-sgRNA5 or ABEmax-sgRNA6 is transfected by electroporation into the cardiac type Fabry disease fibroblasts carrying IVS4+919G>A, and puromycin is used to screen out successfully transfected cells. The base editing conditions are observed through Sanger sequencing. The target site IVS4+919 is indicated by a solid arrow, and the sites with the occurrence of the bystander effect are indicated by hollow arrows. FIG. 4E shows A to G conversion rate analyzed by the next-generation sequencing, and the sequencing depth is approximately 10,000 reads.

FIGS. 5A to 5D show that the restoration of GLA mRNA and GLA protein in the bulk cells of fibroblasts of the cardiac type Fabry disease under the effect of base editing is investigated. Each plasmid of ABEmax-sgRNA5 or ABEmax-sgRNA6 is transfected by electroporation into the fibroblasts of cardiac type Fabry disease carrying IVS4+919G>A, and puromycin is used to screen out successfully transfected cells. FIG. 5A shows the quantitative analyses of normal and aberrant GLA mRNA splicing patterns by quantitative real time polymerase chain reactions, wherein beta-actin is used as the reference for normalization, the expression level of wild-type fibroblasts is set as 1, and the untreated IVS4 cells are used as the control group. FIG. 5B shows the GLA protein expression levels analyzed by western blot, and GAPDH is as the reference for normalization. FIG. 5C shows the quantitative results of GLA protein expression levels, the expression level of wild-type fibroblasts is set as 1, and the untreated IVS4 cells are used as the control group. FIG. 5D shows the detection of cellular GLA enzyme activity, and the enzyme activity of the wild-type fibroblasts is set as 100%, the untreated IVS4 cells are used as the control group. One-way ANOVA is used for analysis, and the differences between each group are analyzed by the Tukey's post-hoc test. The bar graph is presented as mean value ± standard deviation (mean±SD), * means p<0.05. ** means p <0.01. WT: wild type.

FIG. 6 shows that the changes under the effect of base editing are investigated in the bulk cells of fibroblasts of the cardiac type Fabry disease under the effect of base editing. For the bulk cells successfully transfected with ABEmax-sgRNA5 or ABEmax-sgRNA6, the proportions of various gene editing conditions are observed by the next-generation sequencing analysis. WT: wild-type.

FIGS. 7A to 7G show the analysis of the restoration of the GLA gene function in a single cell clone that is accurately corrected back to the wild-type sequence of IVS4+919G under the effect of base editing. FIG. 7A shows that a single cell clone with wild-type+919G which is accurately corrected from the IVS4+919A is screened out from the base-edited bulk cells of the cardiac type Fabry disease, and the target site IVS4+919 is indicated by an arrow. FIG. 7B shows quantitative analysis of normal and aberrant GLA mRNA splicing patterns carried out by quantitative real time polymerase chain reactions, wherein beta-actin is used as the reference for normalization, the expression level of the wild-type fibroblasts is set as 1, and the untreated IVS4 cells are used as the control group. The bar graph is the mean value of triplicate within the group. FIG. 7C shows that the GLA protein expression level is analyzed by western blot, and GAPDH is used as the reference for normalization. FIG. 7D shows the quantitative results of GLA protein expression level. The expression level of wild-type fibroblast cells is set as 1, and the untreated IVS4 cells are used as the control group. FIG. 7E shows the analysis of GLA enzyme activity in a single cell clone. The enzyme activity of the wild-type fibroblasts is set as 100%, and the untreated IVS4 cells are used as the control group. The bar graph represents the mean value of duplicate within the group. FIG. 7F shows Gb3 metabolic wastes stained by immunofluorescence in the single cell clone 6-4. LAMP1 is used as a lysosomal marker, and the cell nuclei are localized with DAPI. In this figure, the visual field is captured by a microscope at 600× magnification. FIG. 7G shows the visual field captured by a microscope at 200× magnification. The fluorescence intensity of Gb3 and the number of nuclei are quantified with Image J. The fluorescence intensity of a single cell is calculated, and the untreated IVS4 cells are used as the control group. One-way ANOVA is used for analysis, and the differences between each group are analyzed by the Tukey's post-hoc test. The bar graph is presented as mean value ± standard deviation (mean±SD), *** means p<0.001. WT: wild type.

FIGS. 8A to 8G show an analysis of the GLA gene function restoration condition of a single cell clone carrying a genotype of IVS4+918_+920GGG under the influence of the bystander effect. FIG. 8A shows that the single cell clone having the genotype of IVS4+918_+920GGG is screened out from the base-edited bulk cells of the cardiac type Fabry disease. The target site IVS4+919 is indicated by a solid arrow, and the sites that generate the bystander effect are indicated by hollow arrows. FIG. 8B shows the quantitative analysis of normal and aberrant GLA mRNA splicing patterns by quantitative real time polymerase chain reactions, wherein beta-actin is used as the reference for normalization, and the expression level of the wild-type fibroblasts is set as 1, and the untreated IVS4 cells are used as the control group. The bar graph represents the mean value of triplicate within the group. FIG. 8C shows the GLA protein expression level analyzed by using western blot, and GAPDH is used as the reference for normalization. FIG. 8D shows the quantitative results of GLA protein expression level, the expression level of the wild-type fibroblasts is set as 1, and the untreated IVS4 cells are used as the control group. FIG. 8E shows the analysis of cellular GLA enzyme activity of a single cell clone. The enzyme activity of the wild-type fibroblasts is set as 100%, and the untreated IVS4 cells are used as the control group. The bar graph represents the mean value of duplicate within the group. FIG. 8F shows that Gb3 metabolic wastes in the single cell clones 2-4, 2-6 and 2-8 are stained by immunofluorescence. LAMP1 is used as a lysosomal marker, and the cell nuclei are localized with DAPI. The visual filed in this figure is captured by a microscope at 600× magnification. FIG. 8G shows the visual field captured by using a microscope at 200× magnification. The fluorescence intensity of Gb3 and the number of nuclei are quantified with Image J. The fluorescence intensity of a single cell is calculated, and the untreated IVS4 cells are used as the control group. One-way ANOVA is used for analysis, and the differences between each group are analyzed by the Tukey's post-hoc test. The bar graph represents the mean value ± standard deviation (mean±SD), * * means p<0.01, *** means p<0.001. WT: wild type; ns: no significant difference.

FIGS. 9A to 9B show the analysis of the GLA gene function restoration conditions that IVS4+920A is corrected to +920G in a single cell clone under the influence of the bystander effect. FIG. 9A shows that a single cell clone carrying a genotype of IVS4+920G is screened out from the base-edited bulk cells of the cardiac type Fabry disease. The site with the occurrence of the bystander effect is indicated by an arrow. FIG. 9B shows the quantitative analysis of normal and aberrant GLA mRNA splicing patterns by quantitative real time polymerase chain reactions, wherein beta-actin is used as the reference for normalization, the expression level of the wild-type fibroblasts is set as 1, and the untreated IVS4 cells are used as the control group. The bar graph represents the mean value of triplicate within the group. WT: Wild-type.

FIG. 10 shows that the off-target effect caused by base editing is investigated in the bulk cells of the cardiac type Fabry disease fibroblasts. The plasmid of ABEmax-sgRNA6 is transfected by electroporation into the cardiac type Fabry disease fibroblasts carrying IVS4+919G>A, and successfully transfected cells are screened out with puromycin (n=3). The A to G conversion rates in 11 potential sequences with the occurrence of the off-target effect are analyzed by next-generation sequencing. The sequencing depth at the on-target site is approximately 10,000 reads, and the sequencing depth at each off-target site is from 66,279 to 261,950 reads.

### DETAILED DESCRIPTION OF THE INVENTION

In the present invention, the gene editing technology is used as a therapeutic strategy, and applied to the fibroblasts of patients suffering from the cardiac type Fabry disease for verification. The experimental results demonstrate the therapeutic potential of the gene editing technology, which can effectively restore the GLA gene function in the cells and achieve a permanent therapeutic efficacy.

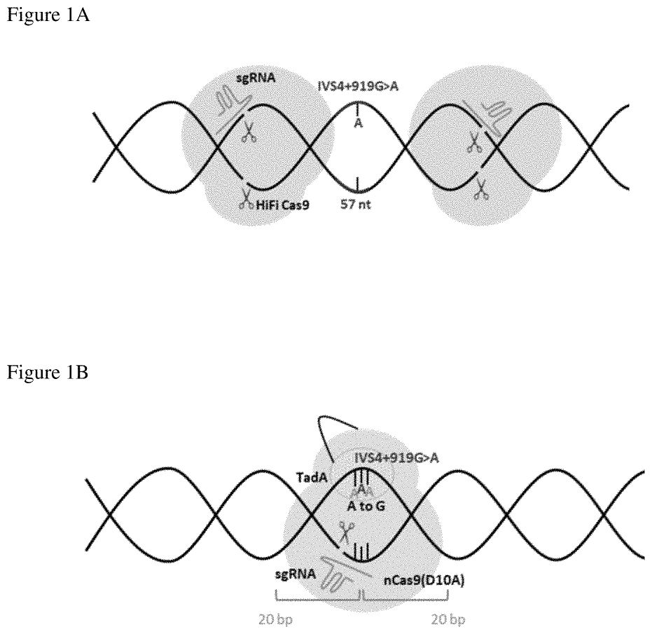

The present invention develops two therapeutic strategies: (1) the first strategy is to modify the sequence of intron 4 in GLA gene through CRISPR/Cas9-mediated nonhomologous end joining (NHEJ), in which suitable single guide RNAs (sgRNAs) are respectively designed upstream and downstream of the GLA IVS4+919G>Apoint mutation, and more novel HiFi Cas9 is used to form accurate DNA double-strand breaks in the target sequence. It allows the aberrantly spliced 57-nucleotide small fragment to undergo double-scissor cleavage in the form of non-homologous end joining, thereby enabling GLA to produce normal mRNA and restore gene function (as shown in FIG. 1A); and (2) the second strategy is through an adenine base editor (ABE), a sgRNA having a length of 20 nucleotides is designed to let the target site GLA IVS4+919G>A able to fall into the working range of sgRNA, making the GLA IVS4+919 G>A point mutation to be corrected accurately. However, the present invention noted that IVS4+918 and IVS4+920 before and after the point mutation in the sequence are As and fall into the base editing window, which may induce bystander effects. In general, the bystander effect is considered to be a condition that must be avoided, because it may lead to unexpected effects. The present invention speculates that the occurrence of the bystander effect at GLA IVS4+918_+920 is highly probable, so the base editing strategy is considered an inappropriate therapeutic strategy to be developed. However, the present invention also observes that IVS4+920 is located in the splicing donor site in the fragment that causes aberrant mRNA splicing, and speculates that the bystander effect may increase the probability of the splicing donor site being destroyed to improve GLA mRNA splicing patterns. According to the experimental results, the present invention overcomes the limitations imposed on the applications of previous base editing system. In addition to correcting the target site IVS4+919G>A, the bystander effect is also used to maximize the benefits of the adenine base editing strategy, thereby facilitating the restoration of gene function (as shown in FIG. 1B).

The delivery tool currently used in gene therapy can be divided into viral vector system and non-viral system, wherein in the viral vector system, the size of the gene that can be carried by an AAV vector is about 4.7 kb. However, the size of the SpCas9 sequence used in the present invention is about 4.1 kb, after the sequences of a promoter, sgRNA and deaminase are added, it will exceed the carrying capacity of AAV. To enable AAV to carry the gene editing system that uses SpCas9 as ribonuclease to infect live animals, the Split-intein system overcomes the limitation of the carrying capacity of AAV. The system can split the sequence of SpCas9 into 5′ end and 3′ end, and they are respectively loaded into two AAV vectors. After N-terminus and C-terminus of SpCas9 protein are respectively translated by the AAV-infected cells, SpCas9 is assembled through the mechanism of protein-splicing under intein to form a full-length functional protein for carrying out gene editing.

In addition to loading the DNA sequence of the gene editing system into a viral vector and delivering it into cell for expression, mRNA or Cas9/sgRNA ribonucleoprotein complexes (RNPs) can also be loaded into a non-viral vector, for example, liposome or lipid nanoparticle. The advantage of this type of delivery system is that gene editing can be carried out instantaneously upon entering the cell. Furthermore, since mRNA and RNPs are quickly decomposed in the cell, the off-target effect is significantly reduced. The above method allows more options for in vivo delivery of the gene editing system, and the gene editing system is expected to be further applied to patients with cardiac type Fabry disease in the future.

In addition to the in vivo delivery of the gene editing system, ex vivo gene editing also provides various treatment methods. In the experiments of the present invention, the fibroblasts carrying IVS4+919 G>Amutation are subjected to gene editing through electroporation, and single cell clones that can successfully restore the GLA gene are screened out. It demonstrates the feasibility of this strategy. Moreover, induced pluripotent stem cells (iPSCs) are promising cell models for in vitro gene editing. Similar to the experimental method of the present invention, in the future, gene editing can be applied to iPSCs cultured from patients suffering from Fabry disease carrying the IVS4+919G>A point mutation. Previous literatures indicated that cardiomyocytes differentiated from iPSCs can repair damaged hearts. Therefore, when the iPSCs of a patient suffering from cardiac type Fabry disease are corrected and differentiated and then injected back into the patient, it is expected that the symptoms of the patient's heart can be improved. Furthermore, CD34+ hematopoietic stem cells (HSPCs) can also be used for correction, and then a patient suffering from Fabry disease can be treated by the method of autologous stem cell transplantation. Previous clinical trials also indicated that the enzyme activity in the patient's plasma could be increased and lyso-Gb3 in blood and urine could be decreased.

The terms “a” or “an” as used herein describe the elements and components of the present invention. This term is used only for convenience of description and to give a basic idea of the present invention. This description should be understood to include one or at least one, and unless it is clear that it is indicated otherwise, the singular also includes the plural. When used in conjunction with the word “comprising” in a claim, the term “a” may mean one or more than one.

The term “or” as used herein in a claim means “and/or,” unless expressly indicated to mean only the other option, or unless the other options are mutually exclusive.

Based on the CRISPR/Cas9 system, the present invention provides a composition, which comprises a polynucleotide, which comprises: (a) a base sequence encoding Cas9 nuclease; (b) a base sequence encoding a first guide RNA which targets GLA gene intron 4 having c.639+919G>A point mutation; and (c) a base sequence encoding a second guide RNA which targets GLA gene intron 4 having c.639+919G>A point mutation, wherein the sequence lengths of the first guide RNA and the second guide RNA are from 17 to 24 nucleotides (nt), and the target sequence targeted by the first guide RNA is an upstream sequence of the c.639+919G>A point mutation, which comprises CTCTGAGAAGAAAATTAAAC (SEQ ID NO: 1) or TCTCAGAGCTCCACACTATT (SEQ ID NO: 2), and the target sequence targeted by the second guide RNA is a downstream sequence of the c.639+919G>Apoint mutation, which comprises TTGACTGTATCTCTCGCATA (SEQ ID NO: 3) or GATACAGTCAAAGTCAGACA (SEQ ID NO: 4).

The point mutation site and the gene loci in the GLA gene sequence are determined with NM_000169 of human GLA as a reference sequence.

When Cas9 and guide RNA are used for CRISPR-based genome editing, sequence-specific cleavage of the genome DNA can be achieved. For example, the nucleic acid encoding a Cas9 nuclease and the nucleic acid encoding a suitable guide RNA can be arranged in separate vectors or arranged together in one single vector to perform in vivo or in vitro knockout or correction of genetic mutations. The short sequence required for Cas9 molecule to bind at appropriate position is called protospacer adjacent motif (PAM), and the sequence adjacent to the 5′ end of PAM is a protospacer sequence. The spacer sequence of its opposite strand and the guide RNA can bind with each other. When expressing the guide RNA and Cas9, the guide RNA is bound with Cas9 as a complex. Cas9 recognizes PAM and the guide RNA complementarily binds with the spacer sequence, so that the Cas9 nuclease generates a splice site at the 5′ end of the protospacer sequence, and double-strand breaks (DSBs) are initiated in the sequence. To repair such breaks, the error-prone non-homologous end joining (NHEJ) mechanism is typically used by cells. The mechanism disrupts the function of target genes through codon insertions or deletions, reading frameshifts, or induces premature termination codons to trigger nonsense-mediated decay.

Since the IVS4+919G>A mutation in the GLA gene causes aberrant mRNA alternative splicing, a 57-nucleotide intronic sequence (c.639+867 to c.639+922) is inserted between exon 4 and exon 5. Therefore, the ranges respectively targeted by those two guide RNAs in the CRISPR system designed by the present invention are the upstream and downstream of the 57-nucleotide sequence in the GLA genome. In one embodiment, the target sequence targeted by the first guide RNA comprises the upstream sequence from c.639+867 to c.639+922 in the GLA genome. In another embodiment, the target sequence targeted by the second guide RNA comprises the downstream sequence from c.639+867 to c.639+922 in the GLA genome. Furthermore, since the c.639+919G>A mutation in the 57-nucleotide intronic sequence (c.639+867 to c.639+922) causes aberrant mRNA splicing, the range targeted by the guide RNA is upstream and downstream of the c.639+919G>A point mutation. In one embodiment, the sequence targeted by the first guide RNA comprises the upstream sequence of the c.639+919G>A point mutation in the GLA genome. In another embodiment, the sequence targeted by the second guide RNA comprises the downstream sequence of the c.639+919G>A point mutation in the GLA genome. Accordingly, the deletion sequence caused by the CRISPR system acting on the GLA gene are required to comprise the c.639+919G>A point mutation.

In one embodiment, the target sequence targeted by the first guide RNA is located in the intronic sequence that is from the c.639+919G>A point mutation toward the 5′ end direction. In another embodiment, the target sequence targeted by the second guide RNA is located in the intronic sequence that is from the c.639+919G>A point mutation toward the 3′ end direction.

In the present invention, the types of the guide RNA comprise a single RNA molecule (single guide RNA, sgRNA) or a combination of two different forms of RNA molecule (comprising tracrRNA and crRNA). In one embodiment, the guide RNA is a single guide RNA.

In the present invention, the Cas9 nuclease includes, but is not limited to, Streptococcus pyogenes Cas9 (SpCas9) nuclease, Staphylococcus aureus Cas9 (SaCas9) nuclease, Streptococcus canis Cas9 (ScCas9) nuclease and variants or derivatives thereof. In one embodiment, the SpCas9 nuclease comprises HiFi Cas9 nuclease.

In some embodiments, the guide RNA guides the Cas9 nuclease to a target sequence, and the guide RNA hybridizes with the target sequence, which can be splitted or cleaved subsequently. The target sequence on which Cas9 works includes the positive and negative strands of genome DNA (that is, the indicated sequence and the opposite strand of this sequence), because the nucleic acid substrate of Cas9 is a double-stranded nucleic acid. Thus, when the guide RNA is complementary to the target sequence, it should be understood that the guide RNA can bind to the opposite strand of the target sequence. In addition, in general design, the sequence of the guide RNA is required to be completely paired with the opposite strand of the target sequence; however, the present invention demonstrates that 1-2 base pairs between the sequence of the guide RNA and the opposite strand of the target sequence are mismatched, which do not affect gene editing efficiency. In one embodiment, the first or the second guide RNA binds to the opposite strand of the target sequence, and the degree of binding is a complete match, or 1-2 base pairs that are mismatched.

Further, in some embodiments, if the nucleotide at the 5′ end of the guide RNA sequence of the present invention is not guanine (G), one or more guanines are added to the 5′ end of the sequence. In some cases, the transcription requires a 5′ G in the design of the guide RNA, which can improve the efficiency of the CRISPR/Cas9 system. In one embodiment, the base at the 5′ end of the sequence of the first or the second guide RNA is guanine.

In one embodiment, the sequence lengths of the first guide RNA and the second guide RNA are from 17 to 20 nucleotides. In a preferred embodiment, the sequence lengths of the first guide RNA and the second guide RNA are 20 nucleotides.

In another embodiment, the sequence of the first guide RNA comprises GTCTGAGAAGAAAATTAAAC (sgRNA1) (SEQ ID NO: 5) or GCTCAGAGCTCCACACTATT (sgRNA2) (SEQ ID NO: 6). The target sequence recognized by sgRNA1 is CTCTGAGAAGAAAATTAAAC (SEQ ID NO: 1), and the target sequence recognized by sgRNA2 is TCTCAGAGCTCCACACTATT (SEQ ID NO: 2). In a preferred embodiment, the sequence of the first guide RNA comprises SEQ ID NO: 6 (sgRNA2).

In one embodiment, the sequence of the second guide RNA comprises GTGACTGTATCTCTCGCATA (sgRNA3) (SEQ ID NO: 7) or GATACAGTCAAAGTCAGACA (sgRNA4) (SEQ ID NO: 8). The target sequence recognized by sgRNA3 is TTGACTGTATCTCTCGCATA (SEQ ID NO: 3), and the target sequence recognized by the sgRNA2 is GATACAGTCAAAGTCAGACA (SEQ ID NO: 4). In a preferred embodiment, the sequence of the second guide RNA comprises SEQ ID NO: 7 (sgRNA3).

An incorrect splice donor site is generated in intron 4 when mutation of G>A occurs at IVS4+919 in the GLA gene, which in turn causes aberrant mRNA alternative splicing, leading to insertion of a fragment of 57-nucleotide intronic sequence between exon 4 and exon 5. Therefore, the first guide RNA targets the upstream of the 57-nucleotide sequence, and the second guide RNA targets the downstream of the 57-nucleotide sequence. The editing is carried out in the GLA gene through the CRISPR/Cas9 system and the 57-nucleotide sequence is disrupted, so that the cell can express normal GLA mRNA. Thus, the GLA gene comprises the 57-nucleotide sequence AGCTCCACACTATTTGGAAGTATTTGTTGACTTGTTACCATGTCTCCCC ACTAAAGT (SEQ ID NO: 9), and the intron 4 sequence between c.639 and c.640 (NM_000169) that is worked by the Cas9 nuclease.

In another embodiment, the polynucleotide further comprises a promoter, which is used for regulating the base sequences encoding the first and the second guide RNA which targets the GLA gene intron 4 having the c.639+919G>A point mutation, or the base sequence encoding the SpCas9 nuclease. In a preferred embodiment, the promoter sequence comprises a U6 promoter. The U6 promoter is used for expressing the base sequences encoding the first and the second guide RNA which targets GLA gene intron 4 having the c.639+919G>A point mutation. In another embodiment, the promoter comprises a CMV promoter. The CMV promoter is used for expressing the base sequence encoding the SpCas9 nuclease.

Furthermore, based on a system of adenine base editor (ABE), the present invention also provides a composition, which comprises a polynucleotide, which comprises: (a) a base sequence encoding a Cas9 nickase; (b) a base sequence encoding a guide RNA which targets GLA gene intron 4 having c.639+919G>A point mutation; and (c) a base sequence encoding a deaminase, wherein the sequence length of the guide RNA is from 17 to 24 nucleotides, the guide RNA targets a target sequence having the c.639+919G>A point mutation, and the c.639+919G>A point mutation in the target sequence corresponds to the working range of the guide RNA.

The locus of c.639+919G>A is determined with NM_000169 of human GLA as a reference sequence.

In the present invention, the Cas9 nickase includes, but is not limited to, SpCas9 nickase, SaCas9 nickase, ScCas9 nickase and variants or derivatives thereof. Conservative amino acids in the nuclease domain of the Cas9 nickase can be substituted to reduce or alter nuclease activity. In some embodiments, the Cas9 nickase can comprise amino acid substitutions in RuvC or RuvC-like nuclease domain. Exemplary amino acid substitutions in the RuvC or RuvC-like nuclease domains include D10A, such as nCas9. In one embodiment, the Cas9 nickase comprises nCas9.

The adenine base editor (ABE) is mainly composed of SpCas9 nickase (for example, nCas9), guide RNA and deaminase, wherein a small nick is formed when nCas9 makes single-strand cleavage in the non-editing strand bound with the guide RNA, and base transversions occur in the target sequence on which sgRNA works, wherein a range in the target sequence is called a base editing window in which the base conversion efficiency is the best. The range of the base editing window varies with the advance of the Cas9 nickase and the base editing system. In ABE7.9, it is defined as the range between the 4th and the 9th base pairs in the target gene in the direction from the 5′ end to the 3′ end; in ABEmax, it is between the 4th and the 7th base pairs; in ABE8e, it is between the 4th and the 8th base pairs; wherein the range of the base editing window of the ABE system that uses SpCas9-VQR(n) is between the 3rd and the 11th base pair. Therefore, the base conversions carried out by deaminase in the base editing window has the highest efficiency to achieve the effect of specific base editing. Finally, the adenine base editor is capable of transverting adenine (A) into guanine (G) in the base editing window. Therefore, in the design of the present invention, the guide RNA targets a target sequence having the c.639+919G>A point mutation, and the c.639+919G>A point mutation in the target sequence falls within the protospacer sequence to which the guide RNA corresponds, and falls within the range between the 3rd and the 11th base pairs in the protospacer sequence in the direction from the 5′ end toward the 3′ end. In one embodiment, the working range of the guide RNA is located between the 3rd to the 11th base pairs in the guide RNA sequence in the direction from the 5′ end toward the 3′ end. In a preferred embodiment, the working range of the guide RNA is located between the 4th and the 9th base pairs in the guide RNA sequence in the direction from the 5′ end toward the 3′ end. In a more preferred embodiment, the working range of the guide RNA is located between the 4th and the 7th base pairs in the guide RNA sequence in the direction from the 5′ end toward the 3′ end.

Since the present invention is to correct the point mutation of c.639+919G>A (IVS4+919G>A), the base sequence encoding the guide RNA (comprising sgRNA5 or sgRNA6) that targets the sequence containing intron 4 c.639+919G>A point mutation in the GLA gene can make the target site IVS4+919G>A fall within the base editing window. As the embodiment of the present invention, sgRNA5 can make the target site locate at the 4th position of the protospacer sequence, while sgRNA6 makes the target site locate at the 5th position. In addition, it is found that ABE can correct As adjacent to IVS4+919 to Gs, which can disrupt the splice donor site to facilitate the restoration of normal GLA mRNA. In one embodiment, in the GLA gene, the target sequence recognized by sgRNA5 comprises CTAAAGTGTAAGTTTCATGA (SEQ ID NO: 10), and the target sequence recognized by sgRNA6 comprises ACTAAAGTGTAAGTTTCATG (SEQ ID NO: 11).

Besides, the sequence of the guide RNA is required to be completely paired with the opposite strand of the target sequence. However, the present invention demonstrates that 1-2 base pairs between the sequence of the guide RNA and the opposite strand of the target sequence are mismatched, which does not affect gene editing efficiency. In one embodiment, the guide RNA binds to the opposite strand of the target sequence, and the degree of binding is a complete pairing, or 1-2 base pairs that are mismatched.

In some embodiments, if the nucleotide at the 5′ end of the guide RNA sequence of the present invention is not a guanine (G), one or more guanines are added to the 5′ end of the sequence. In some cases, the transcription requires 5′ G in the design of the guide RNA, which can improve the efficiency of the ABE system. In one embodiment, the base of the 5′ end of the guide RNA sequence is guanine.

In one embodiment, the sequence length of the guide RNA is from 17 to 20 nucleotides. In a preferred embodiment, the sequence length of the guide RNA is 20 nucleotides.

In another embodiment, the sequence of the guide RNA comprises GTAAAGTGTAAGTTTCATGA (sgRNA5) (SEQ ID NO: 12) or GCTAAAGTGTAAGTTTCATG (sgRNA6) (SEQ ID NO: 13). In a preferred embodiment, the sequence of the guide RNA comprises SEQ ID NO: 13 (sgRNA6).

In one embodiment, the polynucleotide further comprises a promoter, which is used for regulating the base sequence encoding the guide RNA which targets the GLA gene intron 4 having the c.639+919G>A point mutation, the base sequence encoding Cas9 nickase or the base sequence encoding deaminase. In a preferred embodiment, the promoter comprises a U6 promoter. The U6 promoter is used for expressing the base sequence encoding the guide RNA which targets the GLA gene intron 4 having the c.639+919G>A point mutation. In another embodiment, the promoter comprises an EFS promoter. The EFS promoter is used for expressing the base sequence encoding the Cas9 nickase and deaminase.

In the present invention, the polynucleotide further comprises a promoter, which is operably linked to a base sequence encoding the guide RNA or a base sequence encoding Cas9 or deaminase. As used herein, “promoter” means a synthetic or naturally derived molecule capable of conferring, activating or enhancing the expression of a nucleic acid in a cell. A promoter may comprise one or more specific transcriptional regulatory sequences to further enhance expression and/or alter its spatial expression and/or temporal expression. In some embodiments, the promoter can drive and be linked to the upstream of each of the base sequence encoding a guide RNA which targets GLA gene intron 4 having the c.639+919G>Apoint mutation, the base sequence encoding the SpCas9 nuclease, the base sequence encoding the Cas9 nickase and/or the base sequences encoding the deaminase. The promoters that can be linked are not particularly limited, as long as they show promoter activity in the target cell. Examples of the promoters that can be linked to the base sequence encoding the SpCas9 nuclease, the base sequence encoding the Cas9 nickase, or the base sequence encoding the deaminase include, but are not limited to: an EFS promoter, a cytomegalovirus (CMV) promoter, a CK8 promoter, a MHC promoter, a MYOD promoter, an hTERT promoter, a SRalpha promoter, a SV40 promoter, a LTR promoter, a CAG promoter, a Rous sarcoma virus (RSV) promoter and the like. Examples of the promoters that can be linked to the base sequence encoding the guide RNA which targets the GLA gene include, but are not limited to: a U6 promoter, a SNR6 promoter, a SNR52 promoter, a SCR1 promoter, a RPR1 promoter, a U3 promoter, an H1 promoter and a tRNA promoter (which is not a pol III promoter) and the like.

Further, the composition of the present invention further comprises a vector, wherein the vector carries the polynucleotide. Therefore, the polynucleotide having the CRISPR/Cas9 system or the polynucleotide having the ABE system is in the vector. In the present invention, the composition comprises one or more vectors, which respectively carry different base sequence fragments. In one embodiment, the composition comprises a vector, wherein the polynucleotide is in the vector.

In one embodiment, the vector is a plasmid vector, a non-viral vector or a viral vector. When the vector of the present invention is a plasmid vector, the plasmid vector to be used is not particularly limited and can be any plasmid vectors (such as cloning plasmid vectors and expression plasmid vectors). The plasmid vector is prepared by inserting the polynucleotide of the present invention into a plasmid vector by known methods. In a preferred embodiment, the viral vector comprises an adenovirus vector, an adeno-associated virus (AAV) vector, a lentiviral vector, a retrovirus or Sendaivirus vector. In the instant specification, “viral vector” also includes derivatives thereof. Taking the uses in gene therapy into consideration, the AAV vector is preferably used for the reason that transgenes can be expressed over a long period of time, and it is derived from non-pathogenic viruses and is highly safe. The viral vector comprising the polynucleotide of the present invention can be prepared by known methods. In short, a plasmid vector for viral expression inserted with the polynucleotide of the present invention is prepared by transfecting the plasmid into a suitable host cell so as to allow transient production of a viral vector comprising the polynucleotide of the present invention, and then collecting the viral vector. In one embodiment of the present invention, when an AAV vector is used, the serotype of the AAV vector is not particularly limited, and any of AAV1, AAV2, AAV3, AAV4, AAV5, AAV6, AAV7, AAV8, AAV9, AAV10, and variants and the likes thereof can be used. In another embodiment, the non-viral vector comprises a liposome or a lipid nanoparticle.

The present invention further provides a use of a pharmaceutical composition for preparing a drug for treating Fabry disease, wherein the pharmaceutical composition comprises the composition described above.

In the present invention, the pharmaceutical composition comprises a polynucleotide having a CRISPR/Cas9 system or a vector thereof. In addition, the pharmaceutical composition comprises a polynucleotide having an ABE system or a vector thereof.

The composition as used herein can be formulated according to the mode of administration to be used. When the composition is an injectable pharmaceutical composition, it is sterile, pyrogen-free and particulate-free. It is more preferable to use isotonic formulations. In general, isotonic additives include sodium chloride, dextrose, mannitol, sorbitol and lactose. In some cases, an isotonic solution, such as phosphate buffered saline, is preferred. Stabilizers include gelatin and albumin. In some embodiments, a vasoconstrictor is added to the formulation.

The pharmaceutical composition further comprises a pharmaceutically acceptable excipient. The pharmaceutically acceptable excipient can be a functional molecule in the form of a vehicle, an adjuvant, a carrier or a diluent. The pharmaceutically acceptable excipients can be a transfection enhancer, which may include a surfactant, such as immunostimulatory complexes (ISCOMS), an Freund's incomplete adjuvant, including LPS analogs of monophosphoryl lipid A, a muramyl peptide, a quinone analog, a vesicle, such as squalene and squalene, hyaluronic acid, lipids, liposomes, calcium ions, viral proteins, polyanions, polycations or nanoparticles, or other known transfection enhancers.

Furthermore, the present invention provides a method for treating Fabry disease, comprising administering a pharmaceutical composition to a subject suffering from Fabry disease, wherein the pharmaceutical composition comprises the composition described above.

In one embodiment, the pharmaceutical composition comprises a polynucleotide having a CRISPR/Cas9 system or a vector thereof. In another embodiment, the pharmaceutical composition comprises a polynucleotide having an ABE system or a vector thereof.

As used herein, the term “treating” refers to alleviating symptoms or complications; delaying progression of a disease, disorder or condition; relieving or alleviating symptoms and complications; and/or curing or eliminating a disease, disorder or condition.

In some embodiments, the method or use results in gene editing. In some embodiments, the method or use that uses the CRISPR/Cas9 system induces double-strand breaks in a target gene. In some embodiments, the method or use that uses the CRISPR/Cas9 system induces formation of indel mutations during non-homologous end joining of DSBs. In some embodiments, the method or use that uses the CRISPR/Cas9 system induces insertions or deletions of nucleotides in a target gene. In some embodiments, the insertions or deletions of nucleotides in the target gene result in frameshift mutations or premature stop codon to generate non-functional proteins. In some embodiments, the insertions or deletions of nucleotides in the target gene result in blocking or abrogating target gene expression.

In some embodiments, in the method or use using the adenine base editor (ABE), the editor converts A and T base pairs in a target gene to G and C in genome DNA.

As used herein, “gene editing” refers to altering genes. Gene editing includes correcting or restoring a mutant gene. Gene editing may include genetic deletion of a gene (such as a mutant gene or a normal gene). Gene editing can be used to treat diseases by altering related genes.

According to the present invention, the GLA gene of a subject suffering from Fabry disease has intronic splicing mutation IVS4+919G>A, and the point mutation is a point mutation at the nucleotide 919 in the 4th intron (IVS4) in the GLA gene where guanine (G) is transverted to adenine (A). In one embodiment, the GLA gene of the subject suffering from Fabry disease has IVS4+919G>A (c.639+919G>A) point mutation.

In another embodiment, the subject is an animal, preferably a mammal, more preferably a human.

The composition of the present invention comprising the polynucleotide or the vector having the polynucleotide can be administered to the subject by various routes, including oral, parenteral, sublingual, transdermal, rectal, transmucosal, topical, via inhalation, via buccal administration, intrapleural, intravenous, intraarterial, intraperitoneal, subcutaneous, intramuscular, intranasal, intrathecal, and intraarticular, or a combination thereof. In some embodiments, the composition of the present invention is administered to the subject intramuscularly, intravenously, or a combination thereof. The composition can be administered by a conventional syringe, a needle-free injection device, a “microprojectile bombardment gone gun” or other physical methods such as electroporation (“EP”), or ultrasound.

According to the present invention, the dosage of the composition comprising the polynucleotide or the vector having the polynucleotide is not particularly limited, as long as it is an effective amount for treatment. It can be appropriately optimized according to active ingredients, dosage form, age and body weight of the subject, administration schedule, administration method, and the like.

The present invention further provides a method for genetically modifying cells, which comprises delivering the composition into cells ex vivo, wherein the cells ex vivo are from a subject suffering from Fabry disease, and the cells ex vivo comprise GLA gene having c.639+919G>A point mutation.

In the present invention, the pharmaceutical composition comprises a polynucleotide having a CRISPR/Cas9 system or a vector thereof. In addition, the pharmaceutical composition comprises a polynucleotide having an ABE system or a vector thereof.

In one embodiment, the cells ex vivo comprises stem cells. In a preferred embodiment, the stem cells comprise induced pluripotent stem cells (iPSCs) or hematopoietic stem cells. In a more preferred embodiment, the stem cells (such as induced pluripotent stem cells or hematopoietic stem cells) are obtained by culturing cells from a subject suffering from Fabry disease. In another embodiment, the GLA gene of the subject suffering from Fabry disease has IVS4+919G>A (c.639+919G>A) point mutation. In a preferred embodiment, the subject suffering from Fabry disease has c.639+919G>Apoint mutation in the 4th intron of the GLA gene.

The delivery of the composition can be transfection or electroporation of the composition as nucleic acid molecules (such as polynucleotides) that are expressed in the cells and delivered to the cell surface. The nucleic acid molecules can be electroporated by using a BioRad Gene Pulser or an Amaxa Nucleofector IIb Device. Several different buffers can be used, including BioRad Electroporation Solution, Sigma Phosphate Buffered Saline Product #D8537 (PBS), Invitrogen OptiMEM I (OM) or Amaxa Nucleofector Solution V (N.V.). Transfection can comprise a transfection reagent, such as Lipofectamine 2000.

After the composition of the present invention is delivered into the cells, the transfected cells express guide RNA molecules and Cas9 molecules to alter gene expression, or reengineer or alter the genome in the cells. Therefore, the composition of the present invention can be introduced into the cells to genetically correct the GLA gene. Since the cells are induced pluripotent stem cells or hematopoietic stem cells of the subject suffering from Fabry disease, the induced pluripotent stem cells or hematopoietic stem cells are genetically corrected through the composition, and then induced to differentiate into cardiomyocytes for re-implanting into the subject to treat Fabry disease.

The present invention further provides a method for treating Fabry disease, which comprises: (a) isolating or culturing stem cells from a tissue, wherein the tissue is from a subject suffering from Fabry disease, and the stem cells comprise a GLA gene having c.639+919G>A point mutation; (b) delivering the composition into the stem cells, wherein the composition makes gene editing of the c.639+919G>A point mutation in the GLA gene in the stem cells to correct the point mutation; and (c) differentiating the gene-corrected stem cells in step (b) into cardiomyocytes, and administering the cardiomyocytes into the heart of the subject suffering from Fabry disease.

Accordingly, the present invention treats Fabry disease by delivering the polynucleotides having the CRISPR/Cas9 or the ABE gene editing systems. These polynucleotides can be created in a gene construct. As used herein, “gene construct” refers to a DNA or RNA molecule, which comprises a nucleotide sequence encoding a nucleic acid molecule/protein. The encoding sequences include an initiation and a termination signal operably linked to a regulatory element, and the regulatory element includes a promoter and a polyadenylation signal capable of guiding the expression of the administered nucleic acid molecule in the cells of the subject. Thus, the gene construct comprises necessary regulatory element operably linked to the encoding sequences that encode the nucleic acid molecule/protein, so as to express the encoding sequences when present in the cells of the subject.

Thus, the present invention demonstrates that the genome of the cells of the cardiac type Fabry disease are directly corrected by gene editing to be able to achieve a good therapeutic efficiency, and the cells can produce GLA protein enzyme by themselves to achieve a permanent therapeutic efficiency, which can further be applied to a treatment scheme for patients suffering from Fabry disease.

### Examples

The present invention may be embodied in many different forms and should not be construed as limited to the examples set forth herein. The described examples are not intended to limit the scope of the invention as set forth in the claims.

Materials and Methods

Plasmid Construction

The plasmids used in the experiments were all entrusted to and constructed by the Academia Sinica RNAi Core in Taiwan. The used plasmids are shown in Table 1.

TABLE 1
List of plasmids

| Plasmid name | sgRNA sequence (5′→3′) |
|---|---|
| pAAV-CMV-ABEmax-NG-Intein- | None |
| C (EGFP) | |
| pLAS-ABEmax-IVS4-sgRNA5 | 1GTAAAGTGTAAGTTTCATGA (SEQ ID NO: 2) |
| pLAS-ABEmax-IVS4-sgRNA6 | GCTAAAGTGTAAGTTTCATG (SEQ ID NO: 13) |
| SpCas9(HiFi).EGFP-PAC- | sgRNA-2: GCTCAGAGCTCCACACTATT |
| GLA2 + 3 | (SEQ ID NO: 6) |
| | sgRNA-3: GTGACTGTATCTCTCGCATA |
| | (SEQ ID NO: 7) |

Transformation

1 ng of plasmids were taken and added into 30 microliters of ECOS™ 101 competent cells [DH5a] (YB Biotech #FYE678-10VL), mixed gently, placed on ice for 10 minutes, incubated in the dry bath heater at 42° C. for 45 seconds, and then placed on ice for 5 minutes. 1 mL of LB broth was added for recovery, placed it in an incubator at 37° C., and shaken at 240 rpm for 1 hour. Then, it was centrifuged at 3000 rpm for 1 minute, the supernatant was removed and kept about 100 microliters, resuspended well and then added to a LB plate containing ampicillin. After being spreaded evenly, it was incubated at 37° C. for 14 to 16 hours, and a single colony was selected for a large scale production of plasmids.

Large Scale Production of Plasmid DNA

NucleoBond® Xtra Midi Plus (Macherey-Nagel 740412.50) was used in the present invention to manufacture plasmid DNA at a large scale for subsequent uses. A single colony was selected from the LB plate containing ampicillin and added into 3 mL of LB broth containing ampicillin (50 microgram/mL). A small amount of bacterial liquid was shaken and incubated in an incubator at 37° C., 240 rpm for 8 hours. The small amount of bacterial liquid was added into 150 mL of LB broth containing ampicillin (50 microgram/mL) in a conical flask, incubated at 37° C., and shaken at 240 rpm for 14 to 16 hours to manufacture a large amount of bacterial liquid. The large amount of bacterial liquid was centrifuged at 7500 rpm for 15 minutes at 4° C., and then the supernatant was discarded. The bacteria were completely dispersed with 12 mL of RES buffer, 12 mL of LYS buffer was added, gently inverted for 4-5 times, and then allowed it to stand for 5 minutes. 12 mL of NEU buffer was added, and then inverted several times to precipitate the proteins and then poured into a column that was rinsed with 25 mL of EQU buffer in advance. After filtration, the column was rinsed with 8 mL of EQU buffer and the white filter paper was removed. After 8 mL of wash buffer was used for washing, the plasmid DNA was eluted with 5 mL ELU buffer in a 15 mL centrifuge tube. 3.5 mL of isopropanol was added, inverted and mixed until the layers disappeared. A 50-mL syringe filter was used to filter 8.5 mL of liquid, and then the filtrate was filtered again with the syringe filter for avoiding residual plasmid DNA in the filtrate. At this time, the plasmid DNA was adhered to the filter, finally rinsed with 5 mL of 70% EtOH and dried the filter with air. 900 microliters of Tris buffer was used to elute the plasmid DNA into a microcentrifuge tube. After one tenth volume of 3M sodium acetate was added and then evenly mixed, it was centrifuged at 13,200 rpm for 20 minutes at 4° C. At this time, the plasmid DNA precipitated at the bottom of the tube. 1 mL of 70% EtOH was added to wash, centrifuged at 13,200 rpm for 5 minutes at 4° C., and the washing step was repeated once. The alcohol-free microcentrifuge tube was placed at room temperature. After it was air-dried, an appropriate volume of water was added, stood for 1 hour at 37° C. to redissolve, and then stored at −20° C. The plasmid DNA at large scale was confirmed by restriction enzyme digestion reaction.

Agarose Gel Electrophoresis Analysis

Agarose (AMRESCO #0710) and 0.5X TBE buffer were used to prepare 0.8% agarose gel, and 5 microliters/dL SafeView was added during the preparation. 5 microliters of the product of restriction enzyme digestion reaction and 1 microliter of 6X electrophoresis indicator (loading dye) were taken. Then, Bio-1kb™ Mass DNA ladder was used as a marker and loaded to the prepared agarose electrophoresis gel. The electrophoresis was carried out at a voltage of 100 V until DNA fragments were clearly separated, and then the correct fragment size of the agarose gel was confirmed with a UV gel system.

Cell Culture

The culture medium used for human fibroblast cell line of patient with cardiac type Fabry disease and human wild-type fibroblast cell line was the high-glucose Dulbecco's Modified Eagle's Medium (Gibco #12100-100) and contained 10% Fetal Bovine Serum (FBS; Gibco #10437-028), 1% Glutamine (BI #03-020-lb), 1% Sodium pyruvate (BI #03-042-lb) and 1% non-essential amino acids (BI #01-340-1b). The cells were subcultured in a 10-cm culture plate, cultured in a cell incubator at 37° C., 5% CO₂, and then subcultured at a ratio of 1:3 every 2-3 days.

Cell Electroporation

The Ingenio® Electroporation Kits and Solutions (Mirus #MR-MIR50115) were used to perform cell electroporation experiments. Removing culture medium from the human fibroblasts, washing the cells once with PBS solution, and then treating the cells with trypsin at 37° C. for 5 minutes to detach the cells from the culture plate were conducted. Then, the cells were washed from the culture plate and transferred into a centrifuge tube for cell counting. 2 ×105 cells were centrifuged, the supernatant was removed, and 100 microliters of electroporation-specific solution was used for resuspension. After being mixed evenly with 4 micrograms of plasmids, the plasmid-cell mixture was added to the electroporation-specific cuvette. The cuvette was placed in a Lonza Nucleofector™ 2b device, and the electroporation was performed with the program U-030. Finally, the electroporated cells were taken and added into a 12-well culture plate containing 1 ml of cell culture medium for culture. After being transfected for 24 hours, the dead cells were removed, and the cell culture medium was refreshed. Then, the cell adhesions and GFP expressions in the control group were observed to calculate the number of successfully transfected cells. After being transfected for 48 hours, 2 microgram/ml puromycin was used for screening. The cells successfully transfected with the puromycin selection marker would survive. After all the cells in the control group died, the cell culture medium was refreshed, and the cells were cultured for being used in subsequent experiments.

Cellular DNA Extraction

After the cells were detached from the culture plate, the cells were washed from the culture plate and transferred into a centrifuge tube to be centrifuged for 5 minutes (3000 rpm, 4° C.). After the supernatant was removed, PBS was added to wash the cells, and centrifuged for 5 minutes (3000 rpm, 4° C.). After all the supernatant was removed, a QIAamp DNA Mini Kit (QIAGEN #51306) was used to extract DNA from the cells. First, 180 microliters of buffer ATL and 20 microliters of proteinase K were added to the cells, vortexed for about 15 seconds until the cells were completely dispersed, and placed in a dry bath heater at 56° C. for 1 to 3 hours until the cells were completely lysed to release cellular DNA. Then, 200 microliters of buffer AL was added, vortexed for about 15 seconds to mix well, and placed in a dry bath at 70° C. for 10 minutes. Furthermore, 200 microliters of 100% EtOH was added, vortexed for about 15 seconds to mix well, and then all the liquids were transferred to a spin column, centrifuged at 8000 rpm for 1 minute at room temperature and the filtrate was discarded. After 500 microliters of buffer AW1 was added to wash, centrifuged at 8000 rpm for 1 minute at room temperature and the filtrate was discarded. After 500 microliters of buffer AW2 was added to wash, it was centrifuged at the highest speed for 3 minutes at room temperature and the filtrate was discarded. It was centrifuged at the highest speed for 1 minute at room temperature to ensure that the DNA was free of alcohol and residual liquid. The spin column was put on a new microcentrifuge tube, 20 microliters of ddH₂O was added to the spin column and incubated for 5 minutes. Then, it was centrifuged at 8000 rpm for 1 min at room temperature. The obtained filtrate was DNA, and it was stored in a −20° C. refrigerator.

Identification of Cell Genotype

The polymerase chain reaction (PCR) products were performed by Sanger sequencing and next generation sequencing (NGS) to identify the genotype of the cells after gene editing. The primers were designed for the 4th intron (Intron 4) of the GLA gene, which contained the site of ISV4+919. The size of the PCR products obtained by using GLA-IVS4_F and GLA-IVS4_R was 446 bp; and the size of the PCR products obtained by using GLA-IVS4_1F and GLA-IVS4_1R was 232 bp. The related primers are shown in Table 2.

TABLE 2
List of nucleic acid primers

| Primer name | Sequence (5′→3′) |
|---|---|
| GLA-IVS4_F | AGCCCTCTGTCCATTCATTC |
| | (SEQ ID NO: 14) |
| GLA-IVS4_R | CCATATGCGAGAGATACAGTCAA |
| | (SEQ ID NO: 15) |
| GLA-IVS4_1F | TAGGCAGGTGGGATATCAGG |
| | (SEQ ID NO: 16) |
| GLA-IVS4_1R | TTTCTTCTCAGAGCTCCACAC |
| | (SEQ ID NO: 17) |

After the PCR reaction was completed, 1.5% agarose gel was prepared with agarose and 0.5X TBE buffer. The agarose gel electrophoresis was conducted at a voltage of 100 V. After the fragment size was determined to be correct with a UV gel imaging system, sequencing was further used to identify the genotypes.

Off-Target Site Analysis

Cas-OFFinder was used to predict potential off-target sites. The Illumina was used to design nucleic acid primers specifically used for next-generation sequencing (as shown in Table 3), and then the Illumina MiSeq sequencing platform was further used for high-throughput analysis, and finally variant studio software was used to analyze whether or not gene mutation sites were generated.

TABLE 3
List of nucleic acid primers

| Primer name | Sequence (5′→3′) |
|---|---|
| AMPL1558804_U | ATGTAGACACTGTTCTGGAGTCACT |
| | (SEQ ID NO: 18) |
| AMPL1558804_D | ACCAAATGTTACCTTCTCAGTATGT |
| | (SEQ ID NO: 19) |
| AMPL1558819_U | TAGGGTGACAATACAAGAGAAGAGG |
| | (SEQ ID NO: 20) |
| AMPL1558819 D | AGCCTGAACTTATTCATTGTTTCCA |
| | (SEQ ID NO: 21) |
| AMPL1558807_U | CATGTTTATTTCTCCCTTCACTACA |
| | (SEQ ID NO: 22) |
| AMPL1558807 D | GGAGATACAAAAAGATACGTTTCCC |
| | (SEQ ID NO: 23) |
| AMPL1558821_U | GCCAAACGCATATTTCAACTGTAGC |
| | (SEQ ID NO: 24) |
| AMPL1558821 D | GACTACCTCTTGGTGCTATGAGGAA |
| | (SEQ ID NO: 25) |
| AMPL1558817_U | AATGTCACCTTGTCAATGATGTCTC |
| | (SEQ ID NO: 26) |
| AMPL1558817 D | TTTACTGTGTGCTTACTCTCTAAAA |
| | (SEQ ID NO: 27) |
| AMPL1558808_U | AAATGGAATTCTTGCTCTTCCTCAA |
| | (SEQ ID NO: 28) |
| AMPL1558808 D | ATTCATTTTAGGGTGGGTAGCATGT |
| | (SEQ ID NO: 29) |
| AMPL1558805_U | GTGCACCTACTAGAGATTCAACAGT |
| | (SEQ ID NO: 30) |
| AMPL1558805_D | TGGGTCTCAGTTCAAATATGCTTTT |
| | (SEQ ID NO: 31) |
| AMPL1558810_U | TGACCCCTCTATTTCTTATTGAATC |
| | (SEQ ID NO: 32) |
| AMPL1558810 D | TTCAGTGTATACTACTGGCCAAAGG |
| | (SEQ ID NO: 33) |
| AMPL1558820_U | GAGACCTGCTCAATCATTTACTCTG |
| | (SEQ ID NO: 34) |
| AMPL1558820 D | AAAACCAGATTGAGGTAGAACACCT |
| | (SEQ ID NO: 35) |
| AMPL1558816_U | TTGTGCTTATATCCACTCATAGCAA |
| | (SEQ ID NO: 36) |
| AMPL1558816 D | GGCAGTGTGCAAGACTTGGAAATAC |
| | (SEQ ID NO: 37) |
| AMPL1558815_U | AAACAAATGACATTCCTCATGAAGC |
| | (SEQ ID NO: 38) |
| AMPL1558815_D | ATGAGAACATGTGGGCACAGGGAGC |
| | (SEQ ID NO: 39) |

Cellular RNA Extraction

After the cells were detached from the culture plate, the cells were washed from the culture plate and transferred into a centrifugal tube to be centrifuged for 5 minutes (3000 rpm, 4° C.). After removing the supernatant, PBS was added to wash the cells, and it was centrifuged for 5 minutes (3000 rpm, 4° C.). After removing all the supernatant, the Gene-spin Total RNA Purification Kit (Protech #PT-RNA-MS-50) was used to extract RNA from the cells. First, 350 microliters of RNA lysis/2-ME solution was added to the cells, and the centrifugal tube was vortexed for about 15 seconds until the cells were completely lysed to release cellular RNA. After an equal volume of 70% EtOH was added, the centrifugal tube was vortexed for about 15 seconds and mixed evenly, then all of the liquid was transferred into a spin column. It was centrifuged at the highest speed for 1 minute at room temperature, and the filtrate was discarded. After 500 microliters of RNA wash solution 1 was added to wash, it was centrifuged at the highest speed at room temperature for 1 min, and the filtrate was discarded. To ensure that the RNA product was not contaminated by DNA, 80 microliters of DNase I incubation buffer and 2 microliters of DNase I were mixed evenly in advance and then added to the top of the filter membrane of the spin column for reacting at room temperature for 15 minutes. After the reaction was completed, 500 microliters of RNA wash solution 1 was added to wash. It was centrifuged at the highest speed for 1 minute at room temperature, and the filtrate was discarded. After 600 microliters of RNA wash solution 2 was added to wash, it was centrifuged at the highest speed for 1 minute at room temperature, and the filtrate was discarded. The washing step was repeated one more time, and then it was centrifuged at the highest speed for 3 minutes at room temperature to ensure that the RNA was free of alcohol and residual liquids. The spin column was put on a new microcentrifuge tube, and 20 microliters of ddH₂O was added to the spin column. After being incubated for 5 minutes, it was centrifuged at the highest speed for 1 minute at room temperature. The obtained filtrate was RNA, and it was stored at −80° C. refrigerator.

Reverse Transcription Polymerase Chain Reaction

SuperScript III first strand synthesis system (Thermo Scientific #1622) was used, and the extracted cellular RNA was prepared into complementary DNA (cDNA) through reverse transcription polymerase chain reaction. First, 500 ng of RNA and 1 microliter of random hexamer were added into a PCR tube, then nuclease free water was added until the total volume was 12 microliters. It was reacted in a PCR instrument at 65° C. for 5 minutes and then stood at 4° C. After 4 microliters of 5X reaction buffer, 1 microliter of RNase inhibitor, 2 microliters of 10 mM dNTP and 1 microliter of RevertAid M-MuLV RT were added, the total volume was 20 microliters at this time. After mixing evenly, it was reacted in the PCR instrument at 25° C. for 5 minutes →42° C. for 60 minutes→70° C. for 5 minutes and then stood at 4° C. After the reaction was completed, the obtained product was cDNA and it was stored in a −20° C. refrigerator.

Quantitative Real Time Polymerase Chain Reaction (Q-PCR)

The quantitative polymerase chain reaction of different GLA mRNA splicing patterns in the cells were performed with SYBR Green (Roche #04887352001) Kit. Under the normal splicing condition, the nucleic acid primers used in the reaction were GLA-qE4-5 F and GLA-qE5_1R (as shown in Table 4), which were designed for the gap junction of exons 4 and 5 and exon 5 of the GLA gene. The size of the PCR product should be 122 bp. Under the aberrant splicing condition, the nucleic acid primers used in the reaction were GLA-q57nt_F and GLA-q57nt_R (as shown in Table 4), which were designed for the 57-nucleotide fragment that caused aberrant splicing and exon 5. The size of the PCR product should be 162 bp. In addition, Human-beta-actin was used as an endogenous control (as shown in Table 4) in the present invention.

TABLE 4
List of nucleic acid primers

| Primer name | Sequence (5′→3′) |
|---|---|
| GLA-qE4-5_F | CCCTTTCAAAAGCCCAATTA (SEQ ID NO: 40) |
| GLA-qE5_1R | TGGTTAAAAGATGTCCAGTCCA (SEQ ID NO: 41) |
| GLA-q57nt_F | TTGTTACCATGTCTCCCCACT (SEQ ID NO: 42) |
| GLA-q57nt_R | GTCCAGCAACATCAACAATT (SEQ ID NO: 43) |
| Human-beta-actin_184F | AGAGCTACGAGCTGCCTGAC (SEQ ID NO: 44) |
| Human-beta-actin_184R | AGCACTGTGTTGGCGTACAG (SEQ ID NO: 45) |

In the operation step of Q-PCR, SYBR Green (Roche), nucleic acid primers and water were used to prepare the reaction solution. After the appropriate concentration of cDNA was mixed evenly with the reaction solution, the triplicate was performed in a white 96-well plate (LightCycler 480 multiwell).

Formula for calculating the relative expression level: ΔΔCt method, Ct _{target gene}−Ct_{endogenous control}=ΔCt, ΔCt ₛₐₘₚₗₑ−ΔCt _{WT}=ΔΔCt, the relative expression level=2^{-ΔΔCt}

Cell Protein Extraction

Before extracting cell proteins, the protein extraction reagent was prepared. 5 microliters of 20% TritonX-100 (AMRESCO #0694) and 1 microliter of protease inhibitor were added to 994 microliters of RIPA lysis buffer, and then were mixed evenly and placed on ice for subsequent use.

After the cells were detached from the culture plate, the cells were washed from the culture plate and transferred into a centrifuge tube to be centrifuged for 5 minutes (3000 rpm, 4° C.). After the supernatant was removed, PBS was added to wash the cells. It was centrifuged for 5 minutes (3000 rpm, 4° C.) and all the supernatant was removed. An appropriate amount of the prepared protein extraction reagent was added to the cells, vortexed for about 15 seconds until the cells were completely dispersed, and then placed on ice for 30 minutes. During the process, the vortexing was made every 5 minutes to release cellular proteins. Then, it was centrifuged for 10 minutes (13200 rpm, 4° C.), at this time the insoluble cell debris precipitated at the bottom of the tube. After supernatant was taken and transferred into a new microcentrifuge tube, and it was stored in a −80° C. refrigerator.

Protein Quantification

In this experiment, the Bio-Rad protein quantification (Bio-Rad #500-0006) kit was used for protein quantification. First, 10 microliters of albumin from bovine serum (BSA; UR#UR-BSA001) of different concentrations were added to a transparent flat-bottom 96-well plate as standard samples. The concentrations were 0 mg/mL, 0.1 mg/mL, 0.2 mg/mL, 0.3 mg/mL, 0.4 mg/mL, 0.5 mg/mL, respectively, and each concentration was made in duplicate. Then, the protein samples to be quantified were diluted with ddH₂O at an appropriate ratio, and 10 microliters were taken and added into the transparent flat-bottom 96-well plate. Each sample was made in duplicate. After 5 folds of the stock solution of Bio-rad Protein Assay Dye Reagent Concentrate (BCA) was taken and added to sterilized water to be diluted to 1 fold, 190 microliters were taken and added to the transparent flat-bottom 96-well plate containing the samples for reaction. After reacted at room temperature for about 5 minutes, a spectrum analyzer was used for detection under the visible light at a wavelength of 595 nm. A standard curve was established according to the concentrations and absorbance values of the standard samples, and the protein concentrations of the samples were calculated through the standard curve by interpolation.

Enzyme Activity Assay

4-methylumbelliferone-alpha-D-galactoside (Sigma #M7633) was used as the reaction substrate in the present invention. The substrate was used to react with alpha-Gal A enzyme for generating 4-methylumbelliferone (4-MU), which had the property of emitting blue-green fluorescence. N-Acetyl-D-galactosamine (Sigma #A2795) was used to inhibit alpha-Gal B to reduce interference. The activity value of alpha-Gal A enzyme was calculated by detecting its fluorescence intensity.

First, 10 micrograms of the protein to be tested was added in the microcentrifuge tube, ddH₂O was added until the total volume was 5 microliters, and placed on ice after being evenly mixed. Each sample was required to be performed in duplicate. Then, 45 micrograms of a freshly prepared substrate was added to the above microcentrifuge tube and mixed evenly, and placed in a constant temperature incubator at 37° C. for 2 hours. Then, 450 microliters of 0.2N glycine-NaOH solution was added to terminate the reaction. After mixing evenly, 200 microliters was taken into a black flat-bottom 96-well plate. A SpetraMax M5 multi-detection reader was used to read the value of emitted light at the wavelength of 450 nm after using an excitation wavelength of 365 nm. The standard curve was used to calculate the GLA enzyme activity of the protein to be measured.

The standard curve used in the above reaction used 4-methylumbelliferone sodium salt (4-MU; Sigma #M1508-10g) as the standard sample. Serial dilutions (5000, 2500, 1250, 625, 312.5, 156.25, 78.125, 0 nM) were made by diluting the 100 micromoles per liter of 4-MU with 0.2 N glycine-NaOH solution. 200 microliters of each dilution were taken and added into a black flat-bottom 96-well plate. Each concentration was made in duplicate. Then, the SpetraMax M5 multi-detection reader was used to measure the fluorescence intensity of the target sample under the condition of the excitation wavelength of 365 nm/the emission wavelength of 450 nm. The experiment was repeated 3 times to obtain 6 experimental data which the average fluorescence intensity was calculated to establish a standard curve with the concentrations of the standard samples.

The fluorescence reading value of the above target sample, after a blank value was deducted, was substituted into the standard curve formula to obtain the total activity of the target sample protein. The total activity was then substituted into the formula of the protein activity to calculate the enzyme activity value in a fixed unit.

Formula for Calculating Protein Activity

Enzyme activity value (nmol/hr/mg of protein)=(C _{(total activity, nM)}×V_{(total reaction volume, mL)}×10³)/T (reaction time, hr)/protein (total amount of protein, mg)

When the fixed value was substituted into the above formula: enzyme activity value (nmol/hr/mg protein)=(C (_{total activity, nM})×0.5 mL×0.001)/2 hr/0.01 mg

Western Blot

Gel Casting and Electrophoresis

After being wiped clean the transparent glass required for preparing the gel, the transparent glass was arranged in the Mini-PROTEAN® Tetra system (Bio Rad). 7 ml of 10% lower-layer separating gel was added, and isopropanol was used to press the gel surface to be flat. After the lower-layer separating gel solidified, isopropanol was discarded and dried, then 5% of the upper-layer stacking gel was added. A comb was inserted and then waited until the stacking gel was solidified.

The target protein sample was taken, 6X SDS electrophoresis indicator (Loading Dye) (final concentration was 1X) was added, and then mixed well in a microcentrifuge tube. It was heated at 100° C. for 10 minutes to denature the protein, and placed at room temperature for cooling. A protein marker and the target protein were added into a sample chamber for protein electrophoresis analysis. The power supply was set to a fixed voltage of 80 volts for stacking. After the sample ran to the lower-layer separating gel and the protein marker beginning to appear in a separating condition was observed, the fixed voltage was changed to 120 volts until the target protein was clearly separated, i.e., the electrophoresis was completed. The total electrophoresis time was about 2.5 hours.

Transfer and Antibody Staining

After electrophoresis was completed, the Mini-PROTEAN® Tetra system (Bio Rad) was used for blotting. A fixed electric current of 400 milliamperes was used for blotting for 120 minutes. After the blotting was completed, the device was disassembled to remove the PVDF blotting membrane. The PVDF blotting membrane was placed in a clean box and rinsed with deionized water for removing methanol in the transfer buffer to avoid protein precipitation. Then, 5% skim milk/TBST (TBS+0.1% Tween 20) was added for blocking. After shaking for one hour at room temperature to complete the blocking, the PVDF blotting membrane was added with primary antibodies diluted in 5% skim milk/TBST, and shaked at 4° C. to react overnight. The primary antibodies were re-collected the following day, rinsed once with TBST, then the PVDF blotting membrane was placed on a shaker and washed with TBST for 5 minutes. This washing step was repeated six times. The secondary antibodies diluted in 5% of skimmed milk/TBST were added, shaken at room temperature for 1 hour, and rinsed once with TBST. Then, the PVDF blotting membrane was placed on a shaker and washed with TBST for 5 minutes. This washing step was repeated six times to remove unbound-excessive antibodies and non-specific bonding on the blotting membrane. ECL Western Chemiluminescent HRP Substrate was reacted with a chemical luminescence reagent to display color. The signals were detected and recorded by using the UVP BioSpectrum 815 Imaging System.

Immunofluorescence Staining (IFA)

Clean and sterile coverslips were placed on a six-well culture plate, and human fibroblasts were cultured on the coverslip with 2×105 cells per well. After confirming that the cells were adhered to the coverslips the next day, culture was continued until 48 hours. After the cell medium was removed, it was washed with PBS two times, then Cytofix/Cytoperm™ (BD #554722) was used for immunofluorescence staining. 200 microliters of fixation and permeabilization solution containing 4% paraformaldehyde was added, and the fixation was made for 20 minutes. After the cells were fixed and punched, 200 microliters of Perm/wash buffer was added to wash two times, 5 minutes each time. Then, 200 microliters of Perm/wash buffer containing 5% BSA was added for blocking. After reaction at room temperature for 1 hour, the solution on the slide was removed. 200 microliters of primary antibodies mouse anti-Gb3 1:500 (TCI #A2506) and rabbit anti-LAMP1 1:500 (GeneTex #GTX19294) diluted in the Perm/wash buffer containing 2% BSA were added for co-staining, and the reaction was made at 4° C. for overnight. After the primary antibodies were removed in the following day, 200 microliters of Perm/wash buffer solution were added to wash two times, 5 minutes each time. 200 microliters of secondary antibodies Goat anti-mouse antibody (Dylight 488) 1:500 (GeneTex #GTX213111) and Goat anti-rabbit antibody (Dylight 594)1:1000(GeneTex #GTX213110) diluted in Perm/wash buffer containing 2% BSA were added, reacted at room temperature for 1 hour and then removed by suction. 200 microliters of Perm/wash buffer solution were added to wash two times, 5 minutes each time. 200 microliters of DAPI 1:5000 (Sigma #D9542) diluted in Perm/wash buffer solution containing 2% BSA were added for staining for 10 minutes. After 200 microliters of Perm/wash buffer solution was added to wash two times, the observation could be made under a fluorescence microscope after the slides were mounted with a fluorescent mounting medium.

Fluorescence Quantification of Gb3

The slides were placed under a microscope at 200× magnification. After 5-10 visual fields of each slide were photographed, the software Image J was used to quantify the fluorescence intensity of Gb3 and the number of cell nuclei with DAPI. The quantitative results were presented as the fluorescence intensity of a single cell (Gb3 fluorescence/cell), and the average value of 5-10 visual fields was used as the quantitative result of Gb3 fluorescence.

Statistical Analysis

The student's t-test was used to analyze whether there was a significant difference between two groups of experiments. When there were two groups or more, one-way ANOVA was used, and Tukey's post-hoc test was used to analyze the differences between each group. All statistical measures were tested by two-tailed tests. The p value of 0.05 was used to determine the significant level of difference, and GraphPad Prism 5 software was used for statistical analysis.

Results

Strategy 1: Treatment of cell model of cardiac type Fabry disease with IVS4+919G>A by using non-homologous end joining-mediated CRISPR gene editing technology

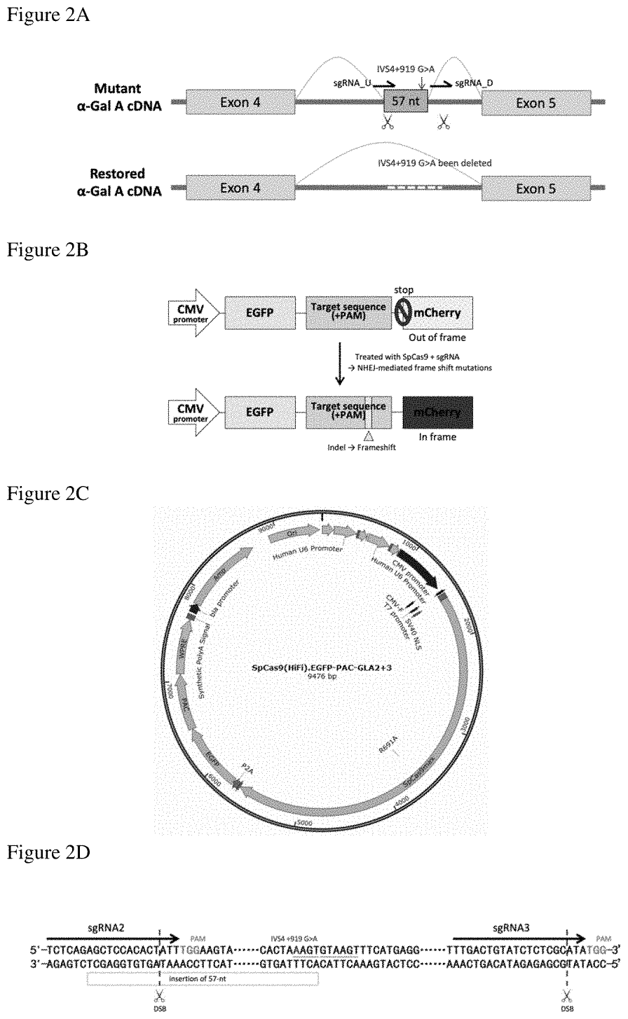

It was known at the time that a mutation of G>A generated at GLA IVS4+919 site would induce an incorrect splicing donor site in intron 4. Furthermore, it caused aberrant mRNA alternative splicing resulted in the insertion of a 57-nucleotide intron sequence between exon 4 and exon 5. Therefore, in the experimental design, the present invention generated double-strand breaks upstream and downstream of the 57-nucleotide sequence that caused aberrant mRNA splicing in the GLA gene sequence through HiFi Cas9 nuclease activity. The present invention speculated that the 57-nucleotide fragment was excised in a non-homologous end-joining manner at the DNA level, and then the cells could express normal GLA mRNA during the process of mRNA splicing (FIG. 2A).

To enable HiFi Cas9 to effectively work on the target sequence, in the present invention, 2 sgRNAs (upstream: sgRNA1 and sgRNA2; downstream: sgRNA3 and sgRNA4) were designed for the upstream and downstream of the GLA IVS4+919G>A point mutation, respectively. Then, a surrogate reporter system was used to test the nuclease cleavage efficiency under the effect of different sgRNAs. The used reporter plasmids comprised the in-frame gene sequence of the EGFP protein on upstream and the out-of-frame gene sequence of the mCherry protein on the downstream. Then, the target sequence (including PAM) targeted by HiFi Cas9 and sgRNA was inserted between the gene sequences of EGFP and mCherry. Under normal condition, only green fluorescence would express. When HiFi Cas9 made a cleavage in the target sequence, sequence indels would probably generate during the process of DNA repair mechanism in the cell. Therefore, the gene sequence of mCherry protein would probably be converted into in-frame, so that the red fluorescence could express (FIG. 2B). The red fluorescence signal was detected through the surrogate reporter system. The results showed that sgRNA2 located upstream of the sequence that caused aberrant mRNA splicing had better nuclease cleavage efficiency (62%) than sgRNA1 (53%), and sgRNA3 located downstream of the sequence had better nuclease cleavage efficiency (76%) than sgRNA4 (58%) (as shown in Table 5).

TABLE 5
sgRNA sequence

| Symbol | NM ID | Species | ID | Sequence | Status | Efficiency |
|---|---|---|---|---|---|---|
| GLA | NM_ | Human | GLA | GTCTGAGAAGAAAAT | OK | 53% |
| | 000169.3 | | sgRNA1 | TAAAC | | |
| | | | | (SEQ ID NO: 5) | | |
| GLA | NM_ | Human | GLA | GCTCAGAGCTCCACA | OK | 62% |
| | 000169.3 | | sgRNA2 | CTATT | | |
| | | | | (SEQ ID NO: 6) | | |
| GLA | NM_ | Human | GLA | GTGACTGTATCTCTCG | OK | 76% |
| | 000169.3 | | sgRNA3 | CATA | | |
| | | | | (SEQ ID NO: 7) | | |
| GLA | NM_ | Human | GLA | GATACAGTCAAAGTC | OK | 58% |
| | 000169.3 | | sgRNA4 | AGACA | | |
| | | | | (SEQ ID NO: 8) | | |

Therefore, a plasmid HiFi Cas9-sgRNA2+3 carrying HiFi Cas9, sgRNA2 and sgRNA3 (FIG. 2C) was constructed for subsequence experiments. It was expected to generate a 97 bp deletion which comprised a fragment that caused aberrant mRNA splicing (FIG. 2D).

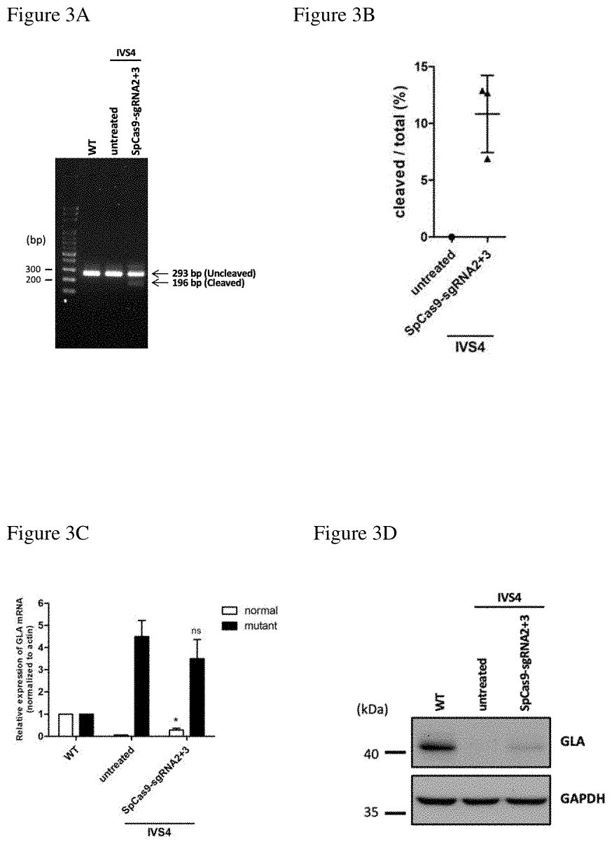

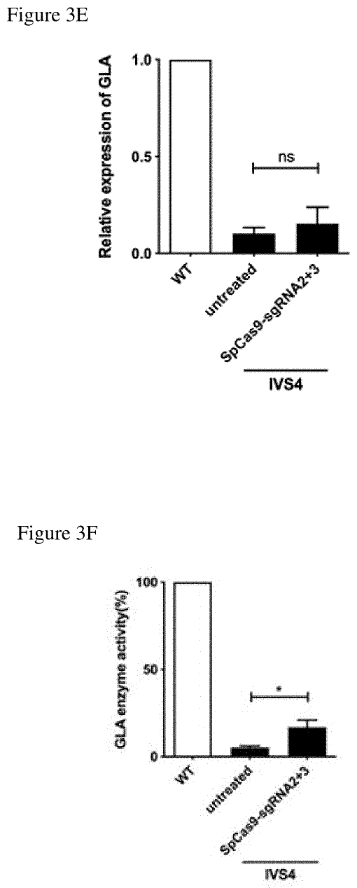

Then, HiFi Cas9-sgRNA2+3 was transfected into the fibroblasts of cardiac type Fabry disease patients by electroporation. 48 hours after the transfection, it was treated with puromycin for 66 hours to select successfully transfected bulk cells. After the DNA was extracted from the successfully transfected bulk cells, the polymerase chain reaction was performed for the GLA intron 4. The size of the PCR products of the wild-type fibroblasts and the untreated fibroblasts of the cardiac type Fabry disease patients observed by agarose gel electrophoresis analysis was 293 bp. In the successfully transfected bulk cells, not only the PCR products of 293 bp but also the occurrence of smaller fragments could be observed, and the size of the fragments was about 196 bp. Therefore, it was speculated that there was indeed gene editing effect in the partial cells of the bulk cells, leading to the excision of the fragments of 97 bp of GLA gene (FIG. 3A). Therefore, in the present invention, gel images were quantitatively analyzed with Image J. The smaller fragments accounted for about 10.8%±3.421% of all PCR products were observed (FIG. 3B). It showed that, under the work of HiFi Cas9 and sgRNA, a non-homologous end-jointing manner indeed caused dual-scissor excision in the DNA fragments that caused aberrant mRNA splicing. Next, in the analytical experiment of GLA mRNA splicing patterns, the present invention designed two sets of primers for normal and aberrant GLA mRNA splicing patterns. Two GLA splicing patterns were quantitatively analyzed through the quantitative real time polymerase chain reaction, and beta-actin was used as the reference for normalization. The results showed that the expression level of normal GLA mRNA in the gene-edited bulk cells was 0.277±0.086, which was significantly higher than the expression level of the untreated fibroblasts of cardiac type Fabry disease patients, 0.06±0.003 (P<0.05). However, the expression level of GLA mRNA generated by aberrant splicing in the gene-edited bulk cells was 3.505±0.856, by the comparison with the expression level 4.492±0.734 in the untreated cells, although there was no statistically significant difference, a downward trend of the abnormal GLA mRNA expression level could still be observed (FIG. 3C).

To further confirm whether or not the expression level of GLA protein could be effectively improved, the western blot was used in the present invention to analyze the expression level of GLA protein, wherein GAPDH was used as the reference for normalization (FIG. 3D). The experimental results revealed that, compared to the expression level of GLA protein in the untreated cells, i.e., 0.101±0.031, the expression level of GLA protein in the gene-edited bulk cells was 0.153±0.085. Although there was no statistically significant difference, partially restored GLA protein expression level could still be observed (FIG. 3E). At the same time, the analysis of the GLA enzyme activity was performed in the present invention, and the wild-type fibroblasts were used as 100% GLA enzyme activity. The results showed that the GLA enzyme activity of the gene-edited cells was 16.843%±4.153%, and the enzyme activity was significantly restored (P<0.05) when compared to the enzyme activity of the untreated cells, 50.193%±0.935% (FIG. 3F).

In summary, through the gene editing strategy mediated by the non-homologous end joining, in the present invention, it was observed that the aberrant GLA mRNA splicing patterns caused by IVS4+919 G>A could be partially corrected. As a result, the normal GLA mRNA splicing patterns significantly increased. In addition to increasing the GLA protein expression level, the GLA enzyme activity also significantly increased, which demonstrated that the therapeutic efficacy could be achieved in the fibroblasts of the cardiac type Fabry disease through the gene editing method that causing deletions of abnormally spliced mRNA fragments.

Strategy 2: Treatment of a Cell Model of Cardiac Type Fabry Disease with IVS4+919G>a by Using Base Editing Technology

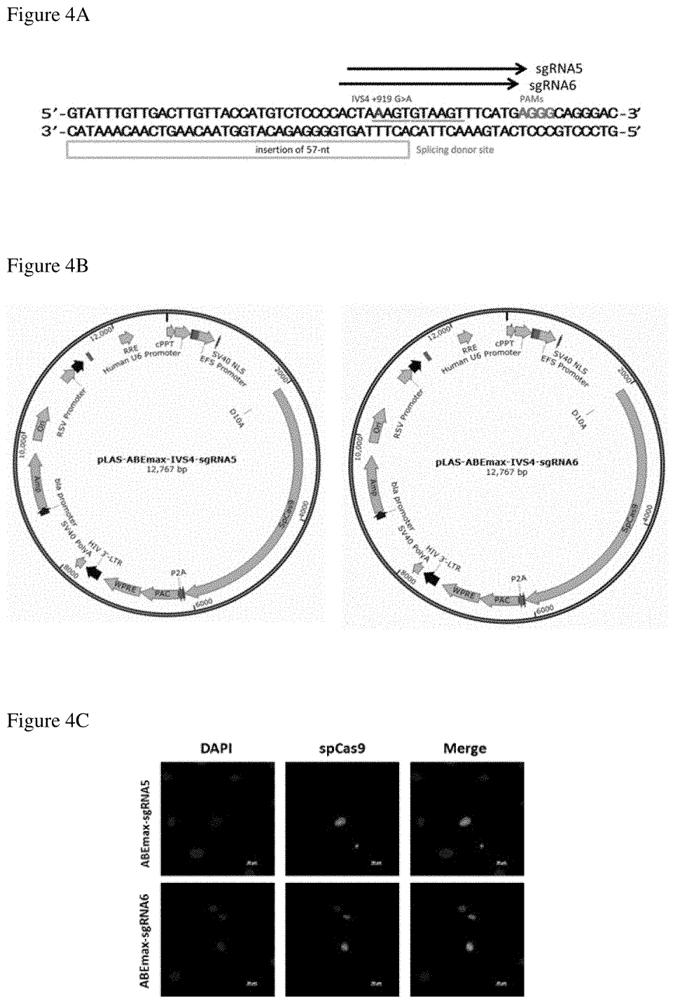

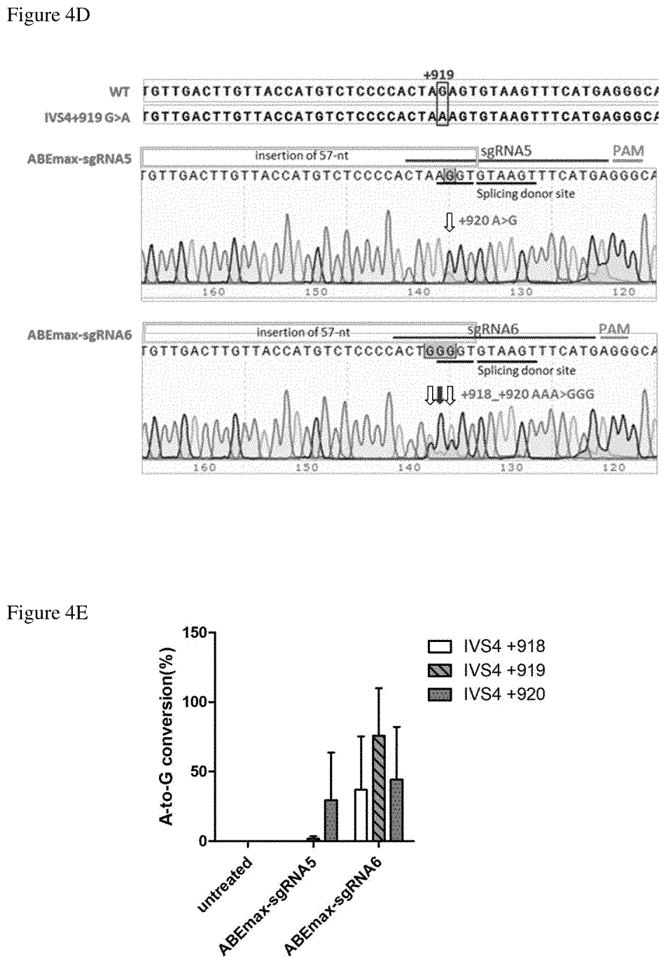

The adenine base editor (ABE) could convert adenine (A) into guanine (G). Therefore, the present invention speculated that the IVS4+919 G>A point mutation could be corrected directly. In the experimental design of the base editing strategy, two sgRNAs were designed in the present invention for making the target site IVS4+919G>A fall into the base editing window. The sgRNA5 could make the target locate at the fourth position of the protospacer sequence, and sgRNA6 made the target locate at the fifth position (FIG. 4A). The sequences of sgRNA5 and sgRNA6 are shown below:

sgRNA5:
(SEQ ID NO: 12)
5′-GTAA₄AGTGTAAGTTTCATGA-3′
sgRNA6:
(SEQ ID NO: 13)
5′-GCTAA₅AGTGTAAGTTTCATG-3′

The underlined portions of the above sequences were the splicing donor sites. The 3′ end of the sgRNA5 could be jointed with a fragment of PAM sequence, namely GGG; and the 3′ end of sgRNA6 could be jointed with a fragment of PAM sequence, namely AGG.

In the present invention, ABEmax-sgRNA5 and ABEmax-sgRNA6 plasmids carrying both the base editor ABEmax and sgRNA were constructed (FIG. 4B). In the present invention, to confirm that the plasmids could work in cells, they were transfected into the fibroblasts of IVS4 cardiac type Fabry disease patients by electroporation. The nCas9 was detected by using immunofluorescence staining, and the cell nuclei were localized with DAPI. The result showed that the expression of nCas9 was indeed observed in the cell nuclei (FIG. 4C).

In the present invention, the plasmids ABEmax-sgRNA5 and ABEmax-sgRNA6 were transfected into the fibroblasts of IVS4 cardiac type Fabry disease patients by electroporation. 48 hours after transfection, it was treated with puromycin for 66 hours to select successfully transfected bulk cells. Through DNA extraction and Sanger sequencing, the results showed that a significant bystander effect was observed in the ABEmax-sgRNA5 group, that is, IVS4+920 (located at the fifth position of the protospacer sequence) had a relatively high proportion of A being converted to G. However, there was no significant base editing observed at +919. Furthermore, under the effect of ABEmax-sgRNA6, in addition to the target site IVS4+919, it was also observed that As at IVS4+918 and +920 (namely from the 4th to the 6th nucleotide of the protospacer sequence) were significantly converted to Gs (FIG. 4D). The efficiency of base editing in the bulk cells was investigated through next-generation sequencing. In the present invention, it was observed that under the effect of ABEmax-sgRNA5, the conversion rate of A to G at IVS4+919 was 1.825%±1.584%, but the average conversion rate of A to G at IVS4+920 was 29.4%±34.227% (FIG. 4E). In addition, it was observed in the present invention that under the effect of ABEmax-sgRNA6, the average of the conversion rate of A to G at the target site IVS4+919 was as high as 75.825%±34.298%, and As at IVS4+918 (36.875% ±38.428%) and +920 (44.325±37.854%) were significantly converted to Gs (FIG. 4E). For the target site IVS4+919, in three experiments, after the effect of ABEmax-sgRNA6, A at the target site IVS4+919 was almost completely converted to G, and the efficiencies were 88.5%, 89.7% and 100%, respectively. The average of the conversion rate of ABEmax-sgRNA6 showed 41.5 times of that of the ABEmax-sgRNA5 group, proving that IVS4+919 could be more accurately corrected by sgRNA6.

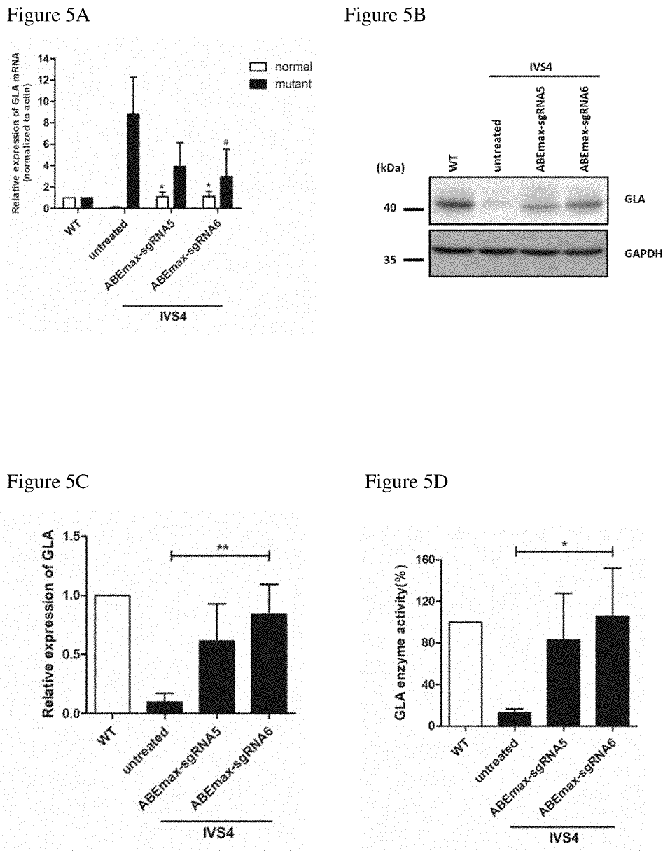

The present invention further investigated whether or not the GLA mRNA splicing patterns in the bulk cells were changed, under the condition that As were converted to Gs at the target site IVS4+919 and the sites IVS4+918 and +920 with the occurrence of significant bystander effects. The normal and aberrant GLA mRNA splicing patterns were quantitatively analyzed through quantitative real time polymerase chain reaction. The present invention observed that after the bulk cells were base edited by ABEmax-sgRNA5, the expression level of the normal GLA mRNA was 1.083±0.414, which was significantly higher than the expression level of the untreated cells, 0.099±0.041 (P<0.05). The expression level of GLA mRNA generated by aberrant splicing was 3.916±2.211. by the comparison with the expression level 8.763±3.503 of the untreated cells, although there was no statistically significant difference, a downward trend of the expression level of abnormal GLA mRNA still could be observed (FIG. 5A). In addition, the expression level of normal GLA mRNA in the bulk cells that were base-edited by ABEmax-sgRNA6 was 1.111±0.494, which was significantly higher than that in the untreated cells (P<0.05). The expression level of GLA mRNA generated by aberrant splicing was 2.968±2.552, which showed a significant downward trend when compared to that in the untreated cells (P<0.05) (FIG. 5A). Particularly, the present invention observed that the target site IVS4+919 and +920 with the occurrence of significant bystander effect were both located at the splicing donor site newly generated by the IVS4+919 G>A mutation (that is the region indicated by underline in FIG. 4A). Therefore, the present invention speculated that though there was a significant bystander effect, the probability of the splicing donor site sequence being disrupted could be increased, which improved the aberrant mRNA alternative splicing caused by the IVS4+919 G>A point mutation and more effectively restored the GLA mRNA splicing patterns. Then, the expression level of GLA protein was analyzed by western blot (FIG. 5B). The expression level of GLA protein in the untreated fibroblasts of the cardiac type Fabry disease patients was 0.097±0.077. The expression level of GLA protein under the effect of ABEmax-sgRNA6 was significantly increased to 0.839±0.251 (P<0.01); and under the effect of ABEmax-sgRNA5, the expression level of GLA protein increased to 0.614±0.317. Although there was no statistically significant difference, a trend of the restoration of the expression level of GLA protein was observed (FIG. 5C). The functions of the GLA protein restored after the effect of base editing were analyzed by GLA enzyme activity, and the GLA enzyme activity of the wild-type fibroblasts was set as 100%. The enzyme activity of the untreated cells was 12.87%±0.3.617%. The results showed that the GLA enzyme activity under the effect of ABEmax-sgRNA5 increased to 82.779%±45.208%. Although there was no statistically significant difference, a significant increase in the GLA enzyme activity could be observed. On the other hand, it was observed that, under the effect of ABEmax-sgRNA6, the GLA enzyme activity was significantly increased to 105.552%±46.355% (P<0.05) (FIG. 5D).

In summary, the base editing strategy not only could effectively correct the IVS4+919 G>A point mutation, and significant bystander effect could also improve the GLA mRNA splicing patterns. It caused that normal GLA mRNA splicing pattern was restored in the fibroblasts of the cardiac type Fabry disease, the GLA protein expression level was effectively increased and its enzyme activity was improved. Therefore, the base editing strategy mediated by the adenine base editor could effectively restore the function of the GLA gene in the fibroblasts of the cardiac type Fabry disease, thereby achieving an excellent therapeutic efficacy.

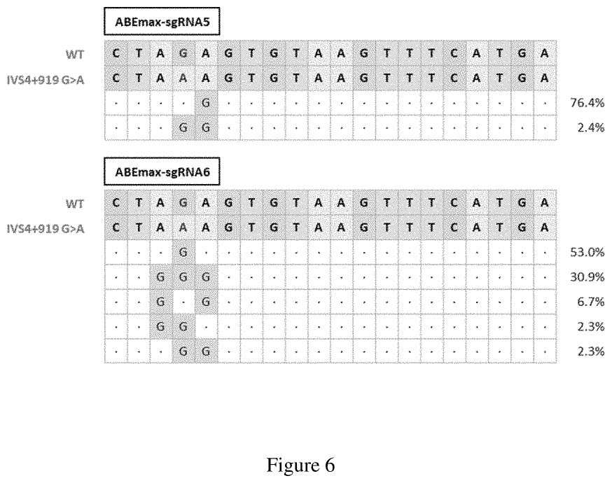

As in three sites, GLA IVS4+918, +919 and +920, had the potential of being converted into Gs, and could effectively restore the function of the GLA gene. Through the next generation sequencing analysis, the present invention observed that the conversion rate of As in these three sites was not all the same, resulting in the bulk cells containing cells with multiple genotypes. It was observed that the proportions of three genotypes of IVS4+919A>G, +920A>G and +918_920AAA>GGG in the bulk cells were relatively high (FIG. 6). Therefore, in the subsequent experiments, single cell clones were used in the present invention to individually verify the GLA gene function of various genotypes.

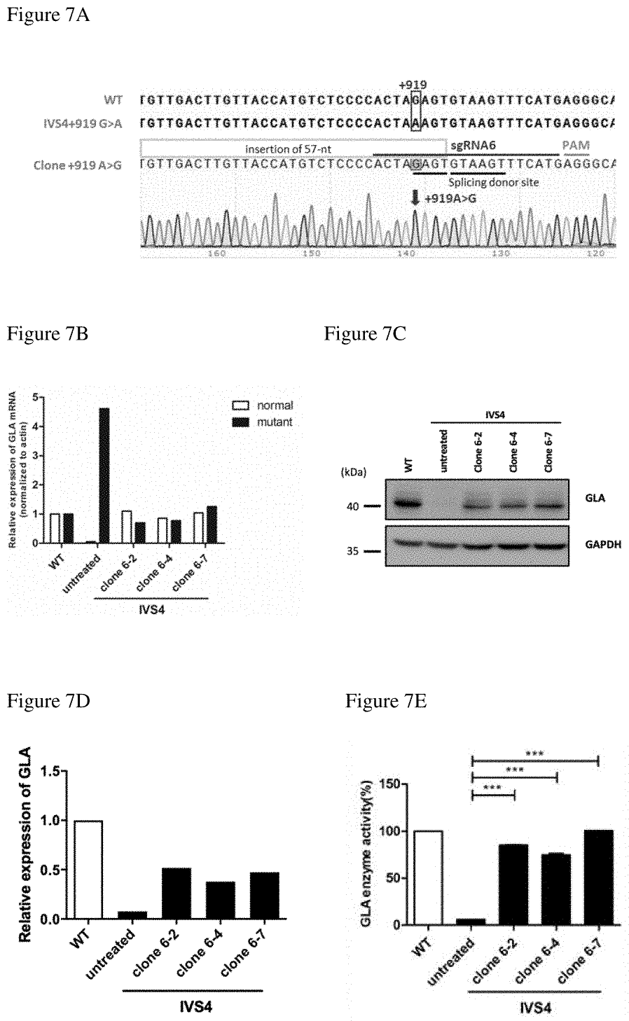

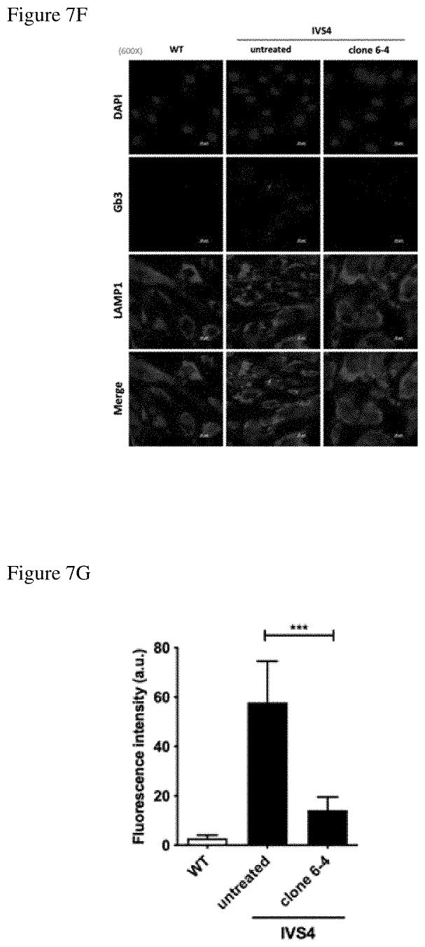

In the present invention, the single cell clone screening was applied to the base-edited bulk cells of the cardiac type Fabry disease, and the selected single cell clones 6-2, 6-4 and 6-7 were cell clones that were accurately corrected back to the wild-type+919G (FIG. 7A). The normal and aberrant GLA mRNA splicing patterns were quantitatively analyzed by quantitative real time polymerase chain reaction. The results showed that the splicing patterns of these single cell clones that were accurately corrected back to the wild genotype by base editing were similar to those of the wild-type fibroblasts (FIG. 7B). The expression level of GLA protein in the single cell clone with IVS4+919A>G was analyzed by western blot. When compared to the untreated cells, it was observed that the expression level of GLA protein increased (FIGS. 7C and 7D), and a trend of significant increase in the GLA enzymatic activity of these single cell clones was further observed (FIG. 7E). Since the GLA protein enzyme was insufficient in the cells, Gb3 was unable to be metabolized normally and accumulated in lysosomes in large amount, leading to cell damage. Accordingly, in the present invention, Gb3 in the single cell clone 6-4 was stained by immunofluorescence. The Gb3 fluorescence intensity (a.u.) of a single cell was quantitatively analyzed, and LAMP1 was used as a lysosomal marker. The results showed that Gb3 significantly accumulated in the lysosomes of the untreated fibroblasts of the cardiac type Fabry disease patients, and the average fluorescence intensity of Gb3 was 57.868±16.726 a.u. However, in the single cell clone 6-4, it was found that Gb3 could be effectively cleared, and the average fluorescence intensity of Gb3 was 14.218±5.308 a.u. (FIGS. 7F and 7G). The above results of the single cell clones showed that, after the effect of the adenine base editor, the IVS4+919G>Apoint mutation could be accurately corrected back to the wild-type sequence, and restored to a state similar to that of the wild-type cells.

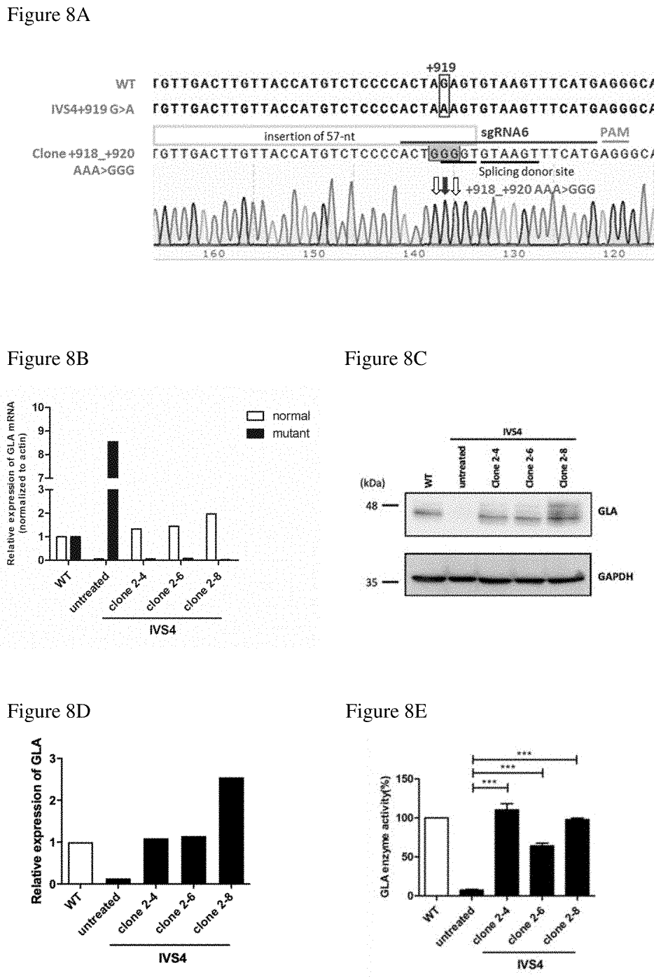

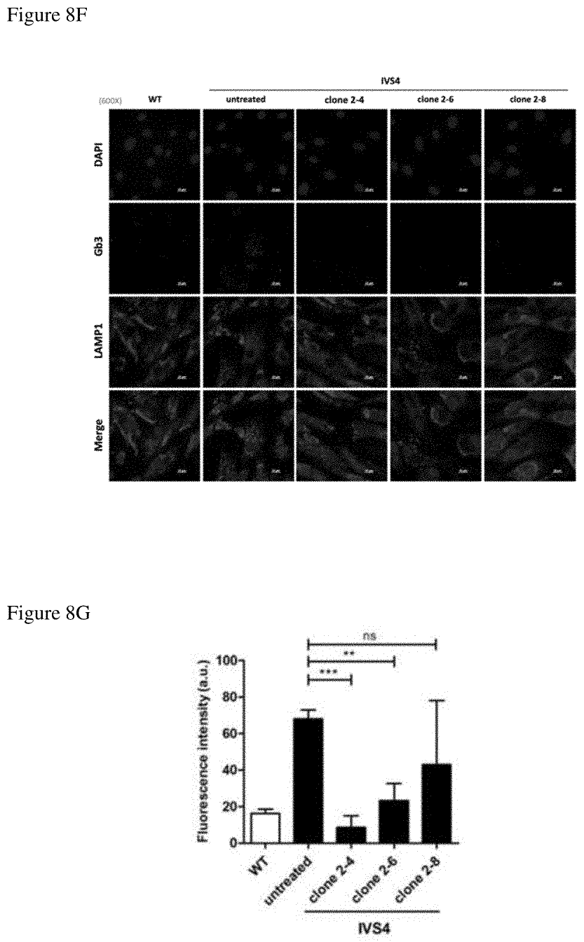

Under the effect of sgRNA6, the present invention found that three consecutive A of IVS4+918 to +920 fell into the base editing window (located at the 4th-6th position of the protospacer sequence). Except for the target site IVS4+919, due to significant bystander effect, As of IVS4+918 and +920 were significantly converted into Gs (FIG. 4C), forming the genotype of +918_+920AAA>GGG. Therefore, the present invention selected the single cell clones 2-2, 2-4, 2-6 and 2-8 with the genotype of +918_+920AAA>GGG (FIG. 8A), and the quantitative real time polymerase chain reaction was used to quantitatively analyze GLA mRNA splicing pattern. It was observed that the expression levels of normal GLA mRNA in these single cell clones with the genotype of +918_+920AAA>GGG were indeed restored, and were from 1.69 to 2.77 times higher than those of the wild-type cells. But the expression levels of abnormal GLA mRNA were not only decreased when compared to that of the SUBSTITUTE SPECIFICATION CLEAN untreated cells, the expression level was from 0.02 to 0.24 times of the wild-type cells, so that it was almost undetectable (Ct values were from 37.09 to 39.26) (FIG. 8B). When compared to the untreated cells, the expression level of GLA protein was also restored (FIGS. 8C and 8D), and the GLA enzyme activity was significantly increased (FIG. 8E). Finally, through immunofluorescence staining, it could be observed that there was significant Gb3 accumulation in the lysosomes of the untreated fibroblasts of the cardiac type Fabry disease patients, and the average fluorescence intensity of Gb3 was 68.158±4.816 a.u. The average fluorescence intensity of Gb3 in a single cell clone 2-4 was significantly decreased to 8.597±6.404 a.u. (P<0.001), and it was significantly decreased to 23.301±9.227 a.u. (P<0.01) in the single cell clone 2-6, which showed that Gb3 could be effectively cleared. In the single cell clone 2-8, the deviation value of Gb3 fluorescence intensity between each visual field tended to be high. Therefore, there was no statistically significant difference as compared to that of the untreated cells. It was speculated that the cell morphological changes might be caused by the limitation of the cell passage number, leading to the beginning of a decline in the ability of GLA protein to clear Gb3. Nevertheless, a downward trend of the average fluorescence intensity of Gb3 was still observed in the single cell clone 2-8, and the value was 42.921±35.196 a.u. (FIGS. 8F and 8G). The results of the above single cell clones showed that, though a significant bystander effect caused by the effect of the base editing system would result in the genotype of +918_+920AAA>GGG, excellent therapeutic efficacy could still be achieved for restoring the GLA gene function to be similar to a state of the wild-type cells.

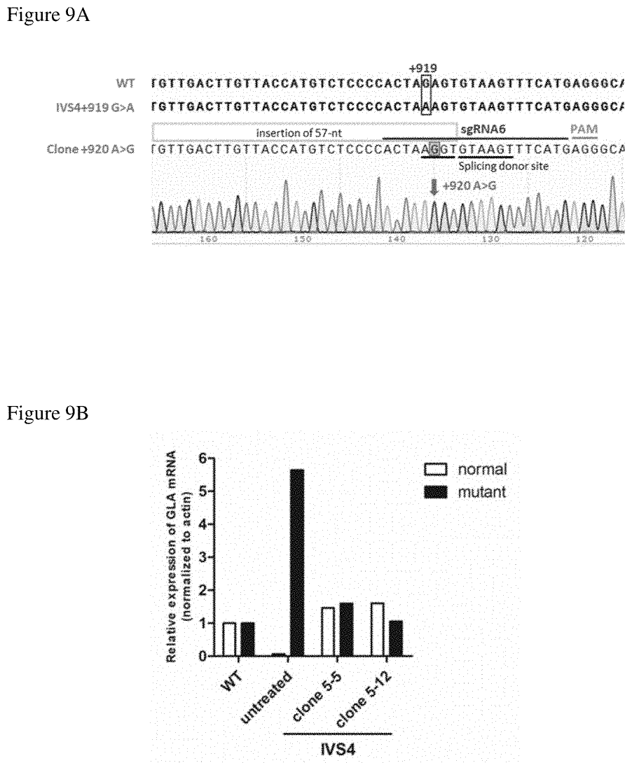

Under the effect of sgRNA5, the present invention found that+920 A was located at the 5th position of the protospacer sequence, and under the effect of sgRNA6, +920 A was located at the 6th position of the protospacer sequence, all falling into the base editing window. In addition, there was a fairly high probability of being corrected to G due to the bystander effect (FIG. 4C), forming the genotype of +920A>G (FIG. 9A). Therefore, through the single cell clones 5-5 and 5-12, the present invention further investigated the effect of the genotype on the restoration of GLA gene function in the cells. The GLA mRNA splicing patterns were quantitatively analyzed through the quantitative real time polymerase chain reaction. It was observed that the expression level of normal GLA mRNA was indeed restored, and the expression level of abnormal GLA mRNA was decreased when compared to that of the untreated cells. The splicing pattern was similar to that of the fibroblasts with wild-type sequence (FIG. 9B). Although the expression level of GLA protein and the level of function restoration of these single cell clones could not be further investigated due to the limitations of passage number, based on the results of the mRNA splicing patterns, the present invention speculated that the genotype of +920A>G could also significantly restore the function of the GLA gene, so that the cells would not be damaged by the accumulations of Gb3, and then restored to the state of normal healthy cells.

In view of the above experimental results, the present invention demonstrated the effects of two gene editing strategies, and found that the adenine base editor had more significant gene editing efficiency and therapeutic efficacy than the CRISPR/Cas9-mediated non-homologous end joining. Furthermore, under the effect of the adenine base editor, the present invention observed that sgRNA6 was more efficient in converting G to A than sgRNA5, and had the ability of correcting the point mutation of +919A>G with high efficiency, and the accompanied bystander effect was beneficial to the restoration of GLA gene function. Since the off-target effect was a hidden concern for gene editing by using the CRISPR/Cas9 system, the present invention selected the ABEmax-sgRNA6 mediated gene editing in the base editing strategy to investigate whether or not off-target effects would occur. The present invention used the online prediction tool CRISPR RGEN Tools to find potential sites in the genome that would occur off-target effects, and then selected the top 11 potential off-target sites. There were 2 mismatched nucleotide base pairs between the sequences having these sites and the sequence of sgRNA6. The gene name and the site of each sequence were analyzed by UCSC (Table 6).

TABLE 6
Sequence having potential off-target site

| | Target sequence of sgRNA + PAM | Gene name | Annotations |
|---|---|---|---|
| On- | A*CTAGAGTGTAAGTTTCATGAGG | GLA | Intron |
| target | (SEQ ID NO: 46) | | (NM_000169.3, Intron 4 |
| | | | of 6) |
| OT-1 | GCTAG*AGTGTAAGTTTG*ATGAGG | AP3B1 | Intron |
| | (SEQ ID NO: 47) | | (NM_001271769, Intron |
| | | | 13 of26) |
| OT-2 | GCTAAAA*TA*TAAGTTTCATGAGG | MACROD2 | Intron |
| | (SEQ ID NO: 48) | | (NM_080676, Intron 5 |
| | | | of 6) |
| OT-3 | GCTAAAA*TGTAAGC*TTCATGAGG | MROH8 | 5’UTR |
| | (SEQ ID NO: 49) | | |
| OT-4 | GCTAAAC*TGTAAGTTC*CATGAGG | RPN2 | Intron |
| | (SEQ ID NO: 50) | | (NM_001135771,, Intron |
| | | | 1 of 16) |
| OT-5 | GCTAAAA*TGTAAGTTTT*ATGAGG | ABL2 | Intron |
| | (SEQ ID NO: 51) | | (NM_007314, Intron 1 |
| | | | of 11) |
| OT-6 | A*CTAC*AGTGTAAGTTTCATGAGG | BANK1 | Intron |
| | (SEQ ID NO: 52) | | (NM_001083907, Intron |
| | | | 2 of 20) |
| OT-7 | A*CTAAAGTGTAAGC*TTCATGAGG | — | Intergenic |
| | (SEQ ID NO: 53) | | |
| OT-8 | GCTAG*AT*TGTAAGTTTCATGAGG | RP11- | Pseudogene |
| | (SEQ ID NO: 54) | 403F21.4 | |
| OT-9 | GCTAAAGTGC*AAGTTTCT*TGAGG | NRG3 | Intron |
| | (SEQ ID NO: 55) | | (NM_001010848, Intron |
| | | | 1 of 8) |
| OT-10 | a*ctac*agtgtaagtttcatgtgg | SHOC2 | Intron |
| | (SEQ ID NO: 56) | | (NM_007373, Intron 2 |
| | | | of 8) |
| OT-11 | GCTG*AAGTGTAAGTTG*CATGAGG | — | Intergenic |
| | (SEQ ID NO: 57) | | |

*: Positions that nucleotide base-pairing mismatches occurred in the potential off-target sequences

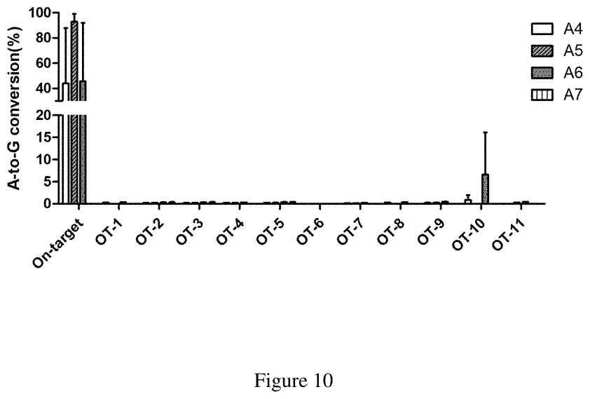

In the bulk cells edited by ABEmax-sgRNA6, next-generation sequencing was used to perform high-throughput sequencing in these 11 potential off-target sites, and the sequencing depth was approximately from 66,279 to 261,950 reads (FIG. 10). Except for OT-10, in the present invention, the conversion of A to G was hardly observed in the other 10 sites. The sequencing results of OT-10 showed that the efficiency of correction of A to G at the 4th position was 0.853%±1.063%, and the efficiency of correction of A to G at the 6th position was 6.587%±90.556%. The corrected site was located in the second intron of SHOC2 gene, which was not recorded in the ClinVar website and dbSNP database. In view of the foregoing, it was proved that ABEmax-sgRNA6 had extremely high specificity to GLA IVS4+919 and a certain level of safety.

In view of the experimental results, the present invention adopted more novel HiFi Cas9 in the traditional CRISPR/Cas9 technology for improving the efficiency of gene editing. The present invention also overcame the application limitations of the base editing system by selecting suitable sgRNAs and applying them to adenine base editors with higher specificity and editing efficiency. Gene editing of the GLA IVS4+919G>A point mutation was performed, and the accompanied bystander effect allowed the strategy to maximize its benefits. It was an important invention to the development of the gene therapy strategy for Fabry disease.

Those who skilled in the art will understand the above concept as a description of the methods used to convey the deposited application information. Those one skilled in the art recognize that these are illustrative only and that many

What is claimed is:

1. A composition, which comprises a polynucleotide, which comprises: (a) a base sequence encoding Cas9 nickase; (b) a base sequence encoding a guide RNA which targets GLA gene intron 4 having c.639+919G>A point mutation; and (c) a base sequence encoding deaminase, wherein the sequence length of the guide RNA is from 17 to 24 nucleutides, and the guide RNA targets a target sequence having c.639+919G>A point mutation, and the c.639+919G>A point mutation in the target sequence corresponds to the working range of the guide RNA.

2. The composition of claim 1, wherein the Cas9 nickase comprises nCas9.

3. The composition of claim 1, wherein the base of the 5′ end of the sequence of the guide RNA is guanine.

4. The composition of claim 1, wherein the sequence of the guide RNA comprises SEQ ID NO: 12 or SEQ ID NO: 13.

5. The composition of claim 1, wherein the working range of the guide RNA is located between the 3rd and the 11th base pairs in the direction from the 5′ end to the 3′ end of the guide RNA sequence.

6. The composition of claim 1, wherein the polynucleotide further comprises a promoter, which is used for regulating the base sequence encoding the guide RNA which targets the GLA gene intron 4 having the c.639+919G>A point mutation, the base sequence encoding the Cas9 nickase or the base sequence encoding the deaminase.

7. The composition of claim 6, wherein the promoter comprises a U6 promoter or an EFS promoter.

8. The composition of claim 1, which further comprises a vector, wherein the polynucleotide is in the vector.

9. The composition of claim 8, wherein the vector comprises a plasmid vector, a non-viral vector or a viral vector.

10. The composition of claim 9, wherein the non-viral vector comprises liposome or lipid nanoparticle.

11. The composition of claim 9, wherein the viral vector comprises an adenoviral vector, an adeno-associated viral vector, a lentiviral vector, a retrovirus or a Sendai virus vector.

12. A method for treating Fabry disease, which comprises administering a pharmaceutical composition to a subject suffering from Fabry disease, wherein the pharmaceutical composition comprises the composition of claim 1.

13. A method for genetically modifying a cell, which comprises delivering the composition of claim 1 into a cell ex vivo, wherein the cell ex vivo is from a subject suffering from Fabry disease, and the cell ex vivo comprises GLA gene having c.639+919G>A point mutation.

14. The method of claim 13, wherein the cell ex vivo comprises stem cells.


SEQUENCE LISTING (57 sequences)

SEQ ID NO: 1 - DNA, 20 bp, Homo sapiens
  source 1..20: mol_type = genomic DNA
ctctgagaagaaaattaaac

SEQ ID NO: 2 - DNA, 20 bp, Homo sapiens
  source 1..20: mol_type = genomic DNA
tctcagagctccacactatt

SEQ ID NO: 3 - DNA, 20 bp, Homo sapiens
  source 1..20: mol_type = genomic DNA
ttgactgtatctctcgcata

SEQ ID NO: 4 - DNA, 20 bp, Homo sapiens
  source 1..20: mol_type = genomic DNA
gatacagtcaaagtcagaca

SEQ ID NO: 5 - RNA, 20 nt, synthetic construct
  source 1..20: mol_type = other RNA; note = single guide RNA
gtctgagaagaaaattaaac

SEQ ID NO: 6 - RNA, 20 nt, synthetic construct
  source 1..20: mol_type = other RNA; note = single guide RNA
gctcagagctccacactatt

SEQ ID NO: 7 - RNA, 20 nt, synthetic construct
  source 1..20: mol_type = other RNA; note = single guide RNA
gtgactgtatctctcgcata

SEQ ID NO: 8 - RNA, 20 nt, synthetic construct
  source 1..20: mol_type = other RNA; note = single guide RNA
gatacagtcaaagtcagaca

SEQ ID NO: 9 - DNA, 57 bp, Homo sapiens
  source 1..57: mol_type = genomic DNA
agctccacactatttggaagtatttgttgacttgttaccatgtctccccactaaagt

SEQ ID NO: 10 - DNA, 20 bp, Homo sapiens
  source 1..20: mol_type = genomic DNA
ctaaagtgtaagtttcatga

SEQ ID NO: 11 - DNA, 20 bp, Homo sapiens
  source 1..20: mol_type = genomic DNA
actaaagtgtaagtttcatg

SEQ ID NO: 12 - RNA, 20 nt, synthetic construct
  source 1..20: mol_type = other RNA; note = single guide RNA
gtaaagtgtaagtttcatga

SEQ ID NO: 13 - RNA, 20 nt, synthetic construct
  source 1..20: mol_type = other RNA; note = single guide RNA
gctaaagtgtaagtttcatg

SEQ ID NO: 14 - DNA, 20 bp, synthetic construct
  source 1..20: mol_type = other DNA; note = forward primer
agccctctgtccattcattc

SEQ ID NO: 15 - DNA, 23 bp, synthetic construct
  source 1..23: mol_type = other DNA; note = reverse primer
ccatatgcgagagatacagtcaa

SEQ ID NO: 16 - DNA, 20 bp, synthetic construct
  source 1..20: mol_type = other DNA; note = forward primer
taggcaggtgggatatcagg

SEQ ID NO: 17 - DNA, 21 bp, synthetic construct
  source 1..21: mol_type = other DNA; note = reverser primer
tttcttctcagagctccacac

SEQ ID NO: 18 - DNA, 25 bp, synthetic construct
  source 1..25: mol_type = other DNA; note = primer
atgtagacactgttctggagtcact

SEQ ID NO: 19 - DNA, 25 bp, synthetic construct
  source 1..25: mol_type = other DNA; note = primer
accaaatgttaccttctcagtatgt

SEQ ID NO: 20 - DNA, 25 bp, synthetic construct
  source 1..25: mol_type = other DNA; note = primer; note = primer
tagggtgacaatacaagagaagagg

SEQ ID NO: 21 - DNA, 25 bp, synthetic construct
  source 1..25: mol_type = other DNA; note = primer
agcctgaacttattcattgtttcca

SEQ ID NO: 22 - DNA, 25 bp, synthetic construct
  source 1..25: mol_type = other DNA; note = primer
catgtttatttctcccttcactaca

SEQ ID NO: 23 - DNA, 25 bp, synthetic construct
  source 1..25: mol_type = other DNA; note = primer
ggagatacaaaaagatacgtttccc

SEQ ID NO: 24 - DNA, 25 bp, synthetic construct
  source 1..25: mol_type = other DNA; note = primer
gccaaacgcatatttcaactgtagc

SEQ ID NO: 25 - DNA, 25 bp, synthetic construct
  source 1..25: mol_type = other DNA; note = primer
gactacctcttggtgctatgaggaa

SEQ ID NO: 26 - DNA, 25 bp, synthetic construct
  source 1..25: mol_type = other DNA; note = primer
aatgtcaccttgtcaatgatgtctc

SEQ ID NO: 27 - DNA, 25 bp, synthetic construct
  source 1..25: mol_type = other DNA; note = primer
tttactgtgtgcttactctctaaaa

SEQ ID NO: 28 - DNA, 25 bp, synthetic construct
  source 1..25: mol_type = other DNA; note = primer
aaatggaattcttgctcttcctcaa

SEQ ID NO: 29 - DNA, 25 bp, synthetic construct
  source 1..25: mol_type = other DNA; note = primer
attcattttagggtgggtagcatgt

SEQ ID NO: 30 - DNA, 25 bp, synthetic construct
  source 1..25: mol_type = other DNA; note = primer
gtgcacctactagagattcaacagt

SEQ ID NO: 31 - DNA, 25 bp, synthetic construct
  source 1..25: mol_type = other DNA; note = primer
tgggtctcagttcaaatatgctttt

SEQ ID NO: 32 - DNA, 25 bp, synthetic construct
  source 1..25: mol_type = other DNA; note = primer
tgacccctctatttcttattgaatc

SEQ ID NO: 33 - DNA, 25 bp, synthetic construct
  source 1..25: mol_type = other DNA; note = primer
ttcagtgtatactactggccaaagg

SEQ ID NO: 34 - DNA, 25 bp, synthetic construct
  source 1..25: mol_type = other DNA; note = primer
gagacctgctcaatcatttactctg

SEQ ID NO: 35 - DNA, 25 bp, synthetic construct
  source 1..25: mol_type = other DNA; note = primer
aaaaccagattgaggtagaacacct

SEQ ID NO: 36 - DNA, 25 bp, synthetic construct
  source 1..25: mol_type = other DNA; note = primer
ttgtgcttatatccactcatagcaa

SEQ ID NO: 37 - DNA, 25 bp, synthetic construct
  source 1..25: mol_type = other DNA; note = primer
ggcagtgtgcaagacttggaaatac

SEQ ID NO: 38 - DNA, 25 bp, synthetic construct
  source 1..25: mol_type = other DNA; note = primer
aaacaaatgacattcctcatgaagc

SEQ ID NO: 39 - DNA, 25 bp, synthetic construct
  source 1..25: mol_type = other DNA; note = primer
atgagaacatgtgggcacagggagc

SEQ ID NO: 40 - DNA, 20 bp, synthetic construct
  source 1..20: mol_type = other DNA; note = forward primer
ccctttcaaaagcccaatta

SEQ ID NO: 41 - DNA, 22 bp, synthetic construct
  source 1..22: mol_type = other DNA; note = reverse primer
tggttaaaagatgtccagtcca

SEQ ID NO: 42 - DNA, 21 bp, synthetic construct
  source 1..21: mol_type = other DNA; note = forward primer
ttgttaccatgtctccccact

SEQ ID NO: 43 - DNA, 20 bp, synthetic construct
  source 1..20: mol_type = other DNA; note = reverse primer
gtccagcaacatcaacaatt

SEQ ID NO: 44 - DNA, 20 bp, synthetic construct
  source 1..20: mol_type = other DNA; note = forward primer
agagctacgagctgcctgac

SEQ ID NO: 45 - DNA, 20 bp, synthetic construct
  source 1..20: mol_type = other DNA; note = reverse primer
agcactgtgttggcgtacag

SEQ ID NO: 46 - DNA, 23 bp, Homo sapiens
  source 1..23: mol_type = genomic DNA
actagagtgtaagtttcatgagg

SEQ ID NO: 47 - DNA, 23 bp, Homo sapiens
  source 1..23: mol_type = genomic DNA
gctagagtgtaagtttgatgagg

SEQ ID NO: 48 - DNA, 23 bp, Homo sapiens
  source 1..23: mol_type = genomic DNA
gctaaaatataagtttcatgagg

SEQ ID NO: 49 - DNA, 23 bp, Homo sapiens
  source 1..23: mol_type = genomic DNA
gctaaaatgtaagcttcatgagg

SEQ ID NO: 50 - DNA, 23 bp, Homo sapiens
  source 1..23: mol_type = genomic DNA
gctaaactgtaagttccatgagg

SEQ ID NO: 51 - DNA, 23 bp, Homo sapiens
  source 1..23: mol_type = genomic DNA
gctaaaatgtaagttttatgagg

SEQ ID NO: 52 - DNA, 23 bp, Homo sapiens
  source 1..23: mol_type = genomic DNA
actacagtgtaagtttcatgagg

SEQ ID NO: 53 - DNA, 23 bp, Homo sapiens
  source 1..23: mol_type = genomic DNA
actaaagtgtaagcttcatgagg

SEQ ID NO: 54 - DNA, 23 bp, Homo sapiens
  source 1..23: mol_type = genomic DNA
gctagattgtaagtttcatgagg

SEQ ID NO: 55 - DNA, 23 bp, Homo sapiens
  source 1..23: mol_type = genomic DNA
gctaaagtgcaagtttcttgagg

SEQ ID NO: 56 - DNA, 23 bp, Homo sapiens
  source 1..23: mol_type = genomic DNA
actacagtgtaagtttcatgtgg

SEQ ID NO: 57 - DNA, 23 bp, Homo sapiens
  source 1..23: mol_type = genomic DNA
gctgaagtgtaagttgcatgagg